SEQUENCE LISTING (5 sequences)

SEQ ID NO: 1 - protein, 7 aa, Artificial sequence
  Synthetic
GGGGDDK

SEQ ID NO: 2 - DNA, 1236 bp, Artificial sequence
  Synthetic
atggtttccaagggcgaagaagacaacatggcgatcatcaaagaatttatgcgttttaaagttcacatggaaggttctgttaacggtcatgagttcgaaattgaaggtgagggtgaaggtcgcccgtacgaaggtacccagaccgcgaaactgaaagttaccaaaggtggtccgctgccgttcgcgtgggacatcctcagcccgcagttcatgtacggttctaaagcgtacgttaaacatccggcggacattccagactacctcaaactctctttccctgaaggtttcaaatgggaacgtgttatgaacttcgaggacggtggtgttgtcaccgttacccaggactcttctctgcaggacggcgagttcatctacaaggtcaaactgcgtggcaccaacttcccgtctgacggtccggttatgcagaaaaaaaccatgggttgggaagcgtcttctgaacgtatgtacccggaagatggtgcgctgaaaggcgaaatcaaacagcgtctgaagctcaaagacggcggtcactacgacgcggaggttaaaaccacctacaaagcgaaaaagccggttcaactgccgggtgcgtacaacgttaatatcaagctggacatcacctctcacaacgaagactacaccatcgttgaacagtacgaacgtgcggaaggccgtcactctaccggtggtatggacgaactgtacaagctcgagctgccggaaaccggtggtggtagtggtggctctggcggttctggtggcagtggcggtagccaagctaaacctcaaattccgaaagataaatcaaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagccaacagatgtagaagttctagatgaacaaaaaggtaaagataaacaattaacattaattacttgtgatgattacaatgaaaagacaggcgtttgggaaaaacgtaaaatctttgtagctacagaagtcaaacatcaccaccatcatcactaa

SEQ ID NO: 3 - DNA, 691 bp, Artificial sequence
  Synthetic
atgaacagcgacagcgagtgcccactgagccacgacggctactgcctgcacgacggcgtgtgcatgtacatcgaggccctggacaagtacgcctgcaactgcgtcgtgggctacatcggcgagcggtgccagtaccgggacctgaagtggtgggagctgagacctcgagctgccggaaaccggtggtggtagtggtggctctggcggttctggtggcagtggcggtagccaagctaaacctcaaattccgaaagataaatcaaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagccaacagatgtagaagttctagatgaacaaaaaggtaaagataaacaattaacattaattacttgtgatgattacaatgaaaagacaggcgtttgggaaaaacgtaaaatctttgtagctacagaagtcaaacatcaccaccatcatcactaa

SEQ ID NO: 4 - DNA, 1413 bp, Artificial sequence
  Synthetic
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatccatgaacagcgacagcgagtgcccactgagccacgacggctactgcctgcacgacggcgtgtgcatgtacatcgaggccctggacaagtacgcctgcaactgcgtcgtgggctacatcggcgagcggtgccagtaccgggacctgaagtggtgggagctgagactcgagctgccggaaaccggtggtggtagtggtggctctggcggttctggtggcagtggcggtagccaagctaaacctcaaattccgaaagataaatcaaaagtggcaggctatattgaaattccagatgctgatattaaagaaccagtatatccaggaccagcaacacctgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagagatgttaagccaacagatgtagaagttctagatgaacaaaaaggtaaagataaacaattaacattaattacttgtgatgattacaatgaaaagacaggcgtttgggaaaaacgtaaaatctttgtagctacagaagtcaaacatcaccaccatcatcactaa

SEQ ID NO: 5 - DNA, 2322 bp, Artificial sequence
  Synthetic
atgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgtgaccccagcccttgtaagaacggagggagctgcacggatctcgagaacagctactcctgtacctgcccacccggcttctacggcaaaatctgtgaattgagtgccatgacctgtgcggacggcccttgctttaacgggggtcggtgctcagacagccccgatggagggtacagctgccgctgccccgtgggctactccggcttcaactgtgagaagaaaattgactactgcagctcttcaccctgttctaatggtgccaagtgtgtggacctcggtgatgcctacctgtgccgctgccaggccggcttctcggggaggcactgtgacgacaacgtggacgactgcgcctcctccccgtgcgccaacgggggcacctgccgggatggcgtgaacgacttctcctgcacctgcccgcctggctacacgggcaggaactgcagtgcccccgtcagcaggtgcgagcacgcaccctgccacaatggggccacctgccacgagaggggccaccgctatgtgtgcgagtgtgcccgaggctacgggggtcccaactgccagttcctgctccccgagctgcccccgggcccagcggtggtggacctcactgagaagctagaggatcgatacgtagagcccagatcttgtgacaaaactcacacatgcccaccgtgcccagcacctgaagccgagggcgcgccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

(19) United States
(12) Patent Application Publication
(10) Pub. No.: US 2024/0042101 A1
(43) Pub. Date: Feb. 8, 2024

(54) PHOTOPATTERNED BIOMOLECULE IMMOBILIZATION TO GUIDE 3D CELL FATE IN NATURAL PROTEIN-BASED HYDROGELS

(71) Applicant: University of Washington, Seattle, WA (US)

(72) Inventors: Cole DeForest, Seattle, WA (US); Ivan Batalov, Seattle, WA (US); Kelly Rose Stevens, Seattle, WA (US)

(73) Assignee: University of Washington, Seattle, WA (US)

(21) Appl. No.: 18/258,735

(22) PCT Filed: Dec. 20, 2021
(86) PCT No.: PCT/US2021/064385

§ 371 Date: Jun. 21, 2023

Related U.S. Application Data
(60) Provisional application No. 63/128,541, filed on Dec. 21, 2020.

(51) Int. Cl. A61L 27/52; C12N 5/00; C07D 207/46

(52) U.S. Cl. CPC A61L 27/52; C12N 5/0068; C07D 207/46; C12N 2537/10

(57) ABSTRACT

Materials, methods, and systems for biorthogonal ligation of hydrogel labels to crosslinked-natural polymer hydrogels are provided. A heterobifunctional linker includes a peptide-reactive activated functional group on the heterobifunctional linker, including an activated amine-reactive functional group, an activated thiol-reactive functional group and being reactive with a hydrogel comprising a crosslinked natural polymer. The heterobifunctional linker also includes a photocaged reactive group including a photocaged hydroxylamine, a photocaged alkoxyamine, a photocaged hydrazide, a photocaged amine, a photocaged tetrazine, or a photocaged alkyne-containing moiety. The peptide-reactive activated functional group does not include an azide.

### CROSS-REFERENCE TO RELATED APPLICATION

This application claims the benefit of U.S. Patent Application No. 63/128,541, filed Dec. 21, 2020, the disclosure of which is incorporated herein by reference in its entirety.

### STATEMENT OF GOVERNMENT LICENSE RIGHTS

This invention was made with government support under Grant Nos. DP2 HL137188 and R35 GM138036, awarded by the National Institutes of Health, and Grant No. DMR 1652141, awarded by the National Science Foundation. The government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 3915-PI 183WOUW.txt. The text file is 12 KB; was created on Dec. 17, 2021 and is being submitted via EFS-Web with the filing of the specification.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a heterobifunctional linker is provided, comprising:
a peptide-reactive activated functional group on the heterobifunctional linker, comprising an activated amine-reactive functional group, an activated thiol-reactive functional group, or any combination thereof, wherein the peptide-reactive activated functional group is reactive with a hydrogel comprising a crosslinked natural polymer; and
a photocaged reactive group on the heterobifunctional linker, comprising a photocaged hydroxylamine, a photocaged alkoxyamine, a photocaged hydrazide, a photocaged amine, a photocaged tetrazine, or a photocaged alkyne-containing moiety,
wherein the peptide-reactive activated functional group does not comprise an azide.

In another aspect, heterobifunctional linker is provided, having a structure:

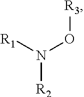

wherein R₁ is selected from a group consisting of an optionally substituted 2-(2-nitrophenyl) propoxycarbonyl moiety, an optionally substituted nitrobenzyl moiety, an optionally substituted coumarin moiety, an optionally substituted boron-dipyrromethene (BODIPY) moiety, and an optionally substituted ruthenium complex;
wherein R₂ is selected from a group consisting of hydrogen, an alkane, an alkyl, and an alcohol; and
wherein R₃ is selected from a group consisting of a N-hydroxysuccinimidide (NHS) activated amine-reactive functional group (e.g., an NHS ester), a N-hydroxysulfosuccinimido (NHSS) activated amine-reactive functional group (e.g., a NHSS ester), an isocyanate, an isothiocyanate, or a maleimide-activated thiol-reactive functional group.

In another aspect, a hydrogel is provided, comprising:
a hydrogel matrix comprising a crosslinked natural polymer; and
a covalently linked hydrogel label at predetermined locations in the hydrogel matrix, wherein the hydrogel label is covalently linked to the hydrogel via a linker.

In another aspect, a hydrogel-labeling kit is provided, comprising:
a heterobifunctional linker comprising a peptide-reactive activated functional group and a photocaged reactive group; wherein the activated functional group does not comprise an azide; and
a hydrogel label activator.

In another aspect, a method of functionalizing a hydrogel matrix is provided, the method comprising:
(a) providing a hydrogel matrix comprising a crosslinked natural polymer and a covalently-linked linker comprising a photocaged reactive group;
(b) selectively exposing the hydrogel matrix to electromagnetic radiation having a predetermined wavelength, at a predetermined time and location in the hydrogel matrix to uncage the reactive group at the predetermined time and the predetermined location; and
(c) reacting the uncaged reactive group with a hydrogel label comprising a complementary reactive group adapted to react with the uncaged reactive group of the covalently-linked linker, to provide a patterned hydrogel.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a block diagram illustrating an example heterobifunctional linker, in accordance with embodiments of the present disclosure.

FIG. 2 is a schematic diagram illustrating an example process for preparing a patterned hydrogel including a covalently linked hydrogel label, in accordance with embodiments of the present disclosure.

FIG. 3A is a schematic diagram illustrating operations of an example process for preparing a patterned hydrogel with an arbitrary pattern formed in two or three spatial dimensions and in a temporal dimension, in accordance with embodiments of the present disclosure.

FIG. 3B is a schematic diagram illustrating an example patterned hydrogel in three spatial dimensions prepared by the example process of FIGS. 2-3A (3D patterning of an anatomical heart model in each natural protein-based gel, showcased with 3D and cross-sectional views. mCherry-CHO is shown in red), in accordance with embodiments of the present disclosure.

FIG. 4A is a reaction schematic illustrating an example reaction for binding the example heterobifunctional linker of FIG. 1 with a natural hydrogel (conjugation of NPPOC—HNO—OSu to primary amines present on the protein-based hydrogel), in accordance with embodiments of the present disclosure.

FIG. 4B is a reaction schematic illustrating an example reaction for uncaging a reactive group of the heterobifunctional linker of FIG. 1 through exposure to electromagnetic radiation (a reactive alkoxyamine is liberated upon photocleavage of the NPPOC cage with cytocompatible radiation (λ=365 nm)), in accordance with embodiments of the present disclosure.

FIG. 4C is a reaction schematic illustrating an example reaction for binding the uncaged reactive group of example heterobifunctional linker of FIG. 1 with a hydrogel label (aldehyde-modified proteins are covalently immobilized within the hydrogel via oxime ligation), in accordance with embodiments of the present disclosure.

FIG. 5A is an example graph presenting normalized brightness data for two labeled natural hydrogels illustrating a concentration effect of example heterobifunctional linker of FIG. 1 (immobilized mCherry fluorescence brightness as a function of NPPOC—HNO—OSu labeling concentration following light treatment (λ=365 nm, 10 mW cm⁻², 10 min), in accordance with embodiments of the present disclosure. Fluorescence brightness values are normalized between 0 (no fluorescence) and 1 (max observed fluorescence for the given experiment). The best-fit solid line assumes first-order photocleavage kinetics of NPPOC and complete oxime ligation. Error bars correspond to ±1 standard deviation about the mean for n=3 experimental replicates.

FIG. 5B is an example graph presenting normalized brightness data for two labeled natural hydrogels illustrating an electromagnetic radiation exposure effect of example heterobifunctional linker of FIG. 1 (immobilized mCherry fluorescence brightness in collagen I and fibrin gels as a function of UV exposure time), in accordance with embodiments of the present disclosure. Fluorescence brightness values are normalized between 0 (no fluorescence) and 1 (max observed fluorescence for the given experiment). The best-fit solid line assumes first-order photocleavage kinetics of NPPOC and complete oxime ligation. Error bars correspond to ±1 standard deviation about the mean for n=3 experimental replicates.

FIG. 6A is an example graph presenting normalized brightness data for a patterned hydrogel illustrating a spatially localized effect of DAPI labelling using example heterobifunctional linker of FIG. 1, in accordance with embodiments of the present disclosure.

FIG. 6B is an example bar graph presenting statistical significance data for the spatially localized effect of DAPI emission in the patterned hydrogel of FIG. 6A, in accordance with embodiments of the present disclosure. Error bars correspond to ±1 standard error about the mean for n≥3 experimental replicates. *p<0.05.

FIG. 6C is an example graph presenting normalized brightness data for a patterned hydrogel illustrating a spatially localized effect of EGFP-EGF labelling using example heterobifunctional linker of FIG. 1, in accordance with embodiments of the present disclosure. Error bars correspond to ±1 standard error about the mean for n≥3 experimental replicates. *p<0.05.

FIG. 6D is an example bar graph illustrating a spatial localization effect in cell fraction data for the patterned hydrogel of FIG. 6A, in accordance with embodiments of the present disclosure. EGFP-EGF-CHO-patterned line regions (+hv) support higher cell counts and higher extents of DNA synthesis than those in the interspaced gap regions (−hv). All analyses were performed on samples six days after seeding. Error bars correspond to ±1 standard error about the mean for n≥3 experimental replicates. *p<0.05.

FIG. 6E is an example bar graph presenting statistical significance data for spatial localization effect in the cell fraction data for the patterned hydrogel of FIG. 6A, in accordance with embodiments of the present disclosure. Error bars correspond to ±1 standard error about the mean for n≥3 experimental replicates. *p<0.05.

FIG. 6F is an example bar graph presenting statistical significance data for spatial localization effect in the cell fraction data for an example hydrogel including control data for the presence of label protein EGF, in accordance with embodiments of the present disclosure. Primary rat hepatocytes exhibited enhanced DNA synthesis w % ben seeded on collagen I gels containing tethered EGF. Quantification of EdU-positive cell fraction indicates statistically significant increase in DNA synthesis for hepatocytes on EGF-modified gels. Error bars correspond to +1 standard error about the mean for n≥3 experimental replicates. *p<0.05.

FIG. 7A is a schematic diagram illustrating an example mechanism for activation of NOTCH enzymes using a natural hydrogel labeled using example heterobifunctional linker of FIG. 1, in accordance with embodiments of the present disclosure. In Notch signaling in U2OS osteosarcoma CSL/luciferase Notch reporter cells, firefly luciferase is expressed in response to the upregulation of Notch signaling caused by the interaction with immobilized Delta-1.

FIG. 7B is a schematic diagram illustrating example luminescence data for a spatially localized NOTCH activation, in accordance with embodiments of the present disclosure. Fibrin gel-encapsulated U2OS Notch reporter cells show localized upregulation of firefly luciferase expression in response to different patterns of immobilized Delta-1-CHO. Half-gel pattern was imaged using Chemidoc XRS+ (BioRad), while 500 μm wide lines and the “W” pattern were imaged using an IVIS Spectrum.

FIG. 7C is a bar graph illustrating statistical significance data for the spatially localized NOTCH activation of FIG. 7B, in accordance with embodiments of the present disclosure. Average luminescence values for functionalized gel regions of each geometry were normalized to 1. Error bars correspond to ±1 standard error about the mean. All analyses were performed on samples seven days after encapsulation/photopatterning. *p<0.001.

FIG. 7D is a schematic diagram illustrating example radiance data for a spatially localized NOTCH activation using a stripe pattern, in accordance with embodiments of the present disclosure. Notch signaling is significantly enhanced for cells within the patterned Delta-1 regions (+hv) compared with that outside (−hv) for each mask geometry.

FIG. 7E is a bar graph illustrating statistical significance data for the spatially localized NOTCH activation of FIG. 7D, in accordance with embodiments of the present disclosure. Average luminescence values for functionalized gel regions of each geometry were normalized to 1. Error bars correspond to ±1 standard error about the mean. All analyses were performed on samples seven days after encapsulation/photopatterning. *p<0.001.

FIG. 7F is a schematic diagram illustrating a line-scan of the example radiance data for a spatially localized NOTCH activation of FIG. 7D, in accordance with embodiments of the present disclosure. Line pattern analysis shows modulation of luciferase expression matching regions of immobilized Delta-1.

FIG. 7G is a schematic diagram illustrating example radiance data for a spatially localized NOTCH activation using an arbitrary two-dimensional mask, in accordance with embodiments of the present disclosure.

FIG. 7H is a bar graph illustrating statistical significance data for the spatially localized NOTCH activation of FIG. 7G, in accordance with embodiments of the present disclosure. Notch signaling is significantly enhanced for cells within the patterned Delta-1 regions (+hv) compared with that outside (−hv) for each mask geometry. Average luminescence values for functionalized gel regions of each geometry were normalized to 1. Error bars correspond to ±1 standard error about the mean. All analyses were performed on samples seven days after encapsulation/photopatterning. *p<0.001.

FIG. 8 is a graph presenting example data for the fraction of modified primary amines on fibrin and collagen gels as a function of NPPOC—HNO—OSu concentration during labeling, in accordance with embodiments of the present disclosure.

FIGS. 9A-C are fluorescence micrographs illustrating mask-based lithographic patterning of mCherry into collagen I (FIG. 9A), fibrin (FIG. 9B), and dECM (FIG. 9C) hydrogels in arbitrary 2D patterns, in accordance with embodiments of the present disclosure. Each gel was labelled with NPPOC—HNO—OSu (300 μM for collagen I, 500 μM for fibrin, 250 μM for dECM) prior to photopatterning (λ=365 nm, 10 mW cm⁻², 3 min) with mCherry-CHO (red). Scale bars=50 μm.

FIGS. 10A-10E are fluorescence micrographs illustrating enhanced hepatocyte proliferation on EGF-functionalized collagen I gels, in accordance with embodiments of the present disclosure. EdU staining is shown in white, nuclei in grey. Scale bars=200 μm. Representative images of primary rat hepatocytes cultured on collagen gels under various conditions: −EGF −hν: negative control, no EGF-CHO added, no UV treatment (FIG. 10A); +EGF −hν: non-specific binding control, EGF-CHO added, no UV treatment (FIG. 10B); +EGF+hν: EGF-CHO added, gels were exposed to UV (k=365 nm, 10 mW cm⁻¹, 3 min) (FIG. 10C); +Commercial EGF: commercially purchased EGF supplemented in culture media, no UV treatment (FIG. 10D); +EGF+hv (10 min): EGF-CHO added, gels were exposed to UV (λ=365 nm, 10 mW cm⁻², 10 min) (FIG. 10E).

FIG. 10F is a bar graph presenting example data from an analysis of EdU-positive cell fraction of the samples, in accordance with embodiments of the present disclosure. Error bars correspond to ±1 standard error about the mean for n=3 experimental replicates.

FIG. 11A is a schematic diagram illustrating evolution of patterned hepatocyte proliferation on EGF-modified collagen I gels, in accordance with embodiments of the present disclosure. Primary rat hepatocytes on collagen I gels patterned with EGFP-EGF-CHO in a 500 μm line pattern, analyzed one and six days after seeding. Confocal images, with DAPI staining in grey and EdU in white. Scale bars=500 μm.

FIG. 11B is a graph presenting example data for image analysis performed across the x axis of the gel to quantify mean DAPI signal in the gels of FIG. 11A, in accordance with embodiments of the present disclosure.

FIG. 11C is a graph presenting example data for image analysis performed across the x axis of the gel to quantify mean Edu+ cell signal in the gels of FIG. 11A, in accordance with embodiments of the present disclosure.

FIG. 12A is a schematic diagram illustrating mask-based lithographic patterning of Delta-1 throughout fibrin gel thickness, in accordance with embodiments of the present disclosure. Delta-1-TAMRA-CHO (0.5 mg mL⁻¹) was patterned within fibrin gels (10 mg mL⁻¹, modified with 200 μM NPPOC—HNO—OSu) through a slitted photomask (500 μm wide line features separated by 500 μm) following methods described in the main text. Confocal microscopy was used to assess relative protein concentration in terms of TAMRA fluorescence throughout the gel. Image taken at a depth of 50 μm. Max observed fluorescence was normalized to a value of 1. Scale bar=200 μm.

FIG. 12B is a graph presenting example data for fluorescence as a function of depth into a UV-treated region corresponding to the mask patterned gel of FIG. 12C, in accordance with embodiments of the present disclosure.

FIG. 13A is a schematic diagram illustrating laser-scanning lithographic patterning of Delta-1 in 3D fibrin gels, in accordance with embodiments of the present disclosure. Employing laser-scanning lithographic patterning, Delta-1-TAMRA-CHO (0.5 mg mL⁻¹) was immobilized ˜200 μm below the fibrin gel (10 mg mL, modified with 200 μM NPPOC—HNO—OSu) surface in the shape of two interlocking rings following methods described in the main text. Confocal microscopy was used to visualize TAMRA fluorophore within the gel. Scale bars=50 μm.

FIG. 13B is a schematic diagram illustrating a second view of patterned 3D fibrin gel of FIG. 13A, in accordance with embodiments of the present disclosure.

### DETAILED DESCRIPTION

Fulfilling a similar role as the extracellular matrix (ECM) in native tissue, gel biomaterials can provide encapsulated cells with mechanical support in a defined geometry, anchors for adhesion and migration, and bioactive chemical signals to guide complex cellular functions (e.g., proliferation, differentiation). Classically, such materials are categorized as “synthetic” or “natural” based on the origin of their underlying components. Synthetic polymer-based gels, including those comprised of poly(ethylene glycol) (PEG) and polyacrylamide, have gained significant traction within the biomaterials community owing to tunable control over their initial network mechanics, chemical composition, and biodegradability. Such systems are synthetically defined, allowing stimuli-responsive functional handles to be installed during formulation, affording exogenously modifiable properties.

Though synthetic gels have been engineered to be responsive to many external stimuli (e.g., pH, temperature, enzyme), electromagnetic radiation, such as biocompatible ultraviolet radiation, permits gels to be modulated spatiotemporally in three-dimensional space and time (also referred to as “4D” in this disclosure) through lithographically defined irradiation. In addition to regulating viscoelastic properties of these materials, photochemical methodologies permit bioactive peptides and proteins to be localized in user-defined patterns within cell-laden gels. Such spatiotemporal control over network biofunctionalization offers the opportunity to impart a simulated tissue with the dynamic biochemical heterogeneity characteristic to native tissues.

Natural protein-based systems, including those derived from fibrin, collagen I, gelatin, or decellularized extracellular matrix (dECM), generally exhibit improved biocompatibility and enhanced matrix remodeling compared with synthetic alternatives. Moreover, natural biomaterials innately provide encapsulated cells with many of the same biochemical cues present in the native extracellular matrix (ECM), giving rise to greater cell-material integration and functional engineered tissue. With the goal of gaining 4D control over natural biomaterial properties, efforts have sought to functionalize protein-based materials with photosensitive moieties.

Installation of alkene functionality (e.g., methacrylate, acrylate) on reaction components has permitted photopolymerization of natural gels, just as chemical crosslinking with photoscissile moieties (e.g., nitrobenzyl) has enabled their photodegradation. These efforts offer spatiotemporal control over the mechanical properties of natural biopolymer-based hydrogels, but fail to permit selective modulation of biochemical aspects of these systems in a user-defined and heterogeneous fashion. There is a need, therefore, for techniques addressed at photo-mediated functionalization of natural gels with bioactive molecules, to solidify natural gels as choice materials for fundamental cell studies and translational applications.

Prior efforts at photoimmobilization of proteins within natural hydrogels relied on a sortase transpeptidase-mediated coupling of a chemically tagged streptavidin onto a photo-uncaged polyglycine peptide grafted to a backbone material. In the prior work, biotinylated proteins of interest were noncovalently immobilized with spatial control within natural gels. The application of the prior work involved controlled axon guidance using Nerve Growth Factor within an engineered hyaluronan-based matrix. In this way, photopatterned cell function on or within natural protein-based biomaterials was not demonstrated.

Photomediated oxime ligation represents a chemical framework for photopatterning of synthetic polymer-based materials that can be used to spatially modify natural gels with unexpected benefits associated with covalent protein immobilization, chemical accessibility, design simplicity, and overall ease of use. As a bioorthogonal reaction that can be photochemically triggered, embodiments described herein demonstrate chemistry that is uniquely capable of spatiotemporally regulating cell function on/in protein-based gels. Advantageously, such spatiotemporal control enables arbitrary and/or user-defined control over when and where ligation occurs with high specificity in the presence of living systems. Embodiments described herein relate to a generalizable strategy for covalent decoration of naturally derived biomaterials with bioactive proteins including growth factors, to spatially control encapsulated cell fate.

In an illustrative example, described in more detail in reference to the Examples below, some embodiments of the present disclosure take advantage of primary amines ubiquitously present on proteins (both at N-terminus and on lysine side chains) by functionalizing natural gel precursors with a 2-(2-nitrophenyl) propoxycarbonyl (NPPOC)-photocaged alkoxyamine (H2NO—) through reaction with an activated N-Hydroxysuccinimide ester (—OSu). In reference to the —OSu reaction, the approach implicates a chemistry exhibiting comparatively long-term reagent stability and synthetic accessibility.

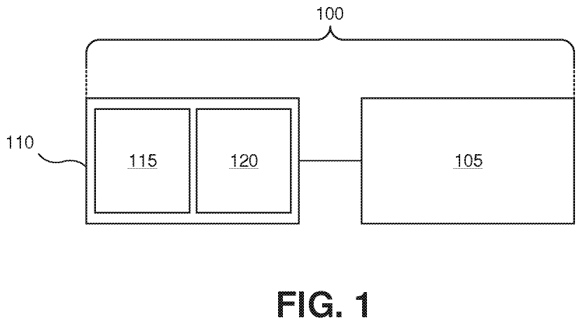

The resulting trifunctional small molecule, as described in more detail in reference to FIG. 1, can be produced on gram scale through readily accessible chemistry and used to modify/functionalize a wide variety of natural biomaterials. Upon exposure of the functionalized gel to cytocompatible near-UV photons having a wavelength from about 200 nm to about 400 nm (e.g., λ=365 nm), the photocage (e.g., NPPOC) is cleaved in a dose-dependent manner, liberating the photocaged alkoxyamine and permitting localized condensation with aldehyde-functionalized proteins.

As such, mask-based and/or laser-scanning lithographic activation of this photomediated oxime ligation permits 4D control over protein immobilization within natural biopolymer-based hydrogels. Advantageously, the techniques described herein permit natural biopolymer-based hydrogels to pattern two responses that have not been achieved even in purely synthetic materials. A first response is 2D primary rat hepatocyte proliferation on collagen gels using immobilized epidermal growth factor (EGF). A second response is 3D U2OS NOTCH signaling activation within fibrin gels decorated with tethered Delta-1.

FIG. 1 is a block diagram illustrating an example heterobifunctional linker 100, in accordance with embodiments of the present disclosure. Example heterobifunctional linker 100 is illustrated functionally, to demonstrate the relative relationships between different orthogonally mediated reactive functional groups. The role of each functional group described in reference to the forthcoming figures and examples derive from the chemical structure of the functional groups of example heterobifunctional linker 100, which significantly improves spatio-temporal localization of target molecules in a biocompatible matrix. Applications of example heterobifunctional linker 100 include but are not limited to guiding complex cellular function (e.g., proliferation, differentiation) through electromagnetic radiation-modulated binding of natural-protein based hydrogels with bio-active molecules. In this context, the term “guiding” is used to indicate intentional indication of favorable regions and unfavorable regions of a three-dimensional material for cell migration and/or growth in both time and space.

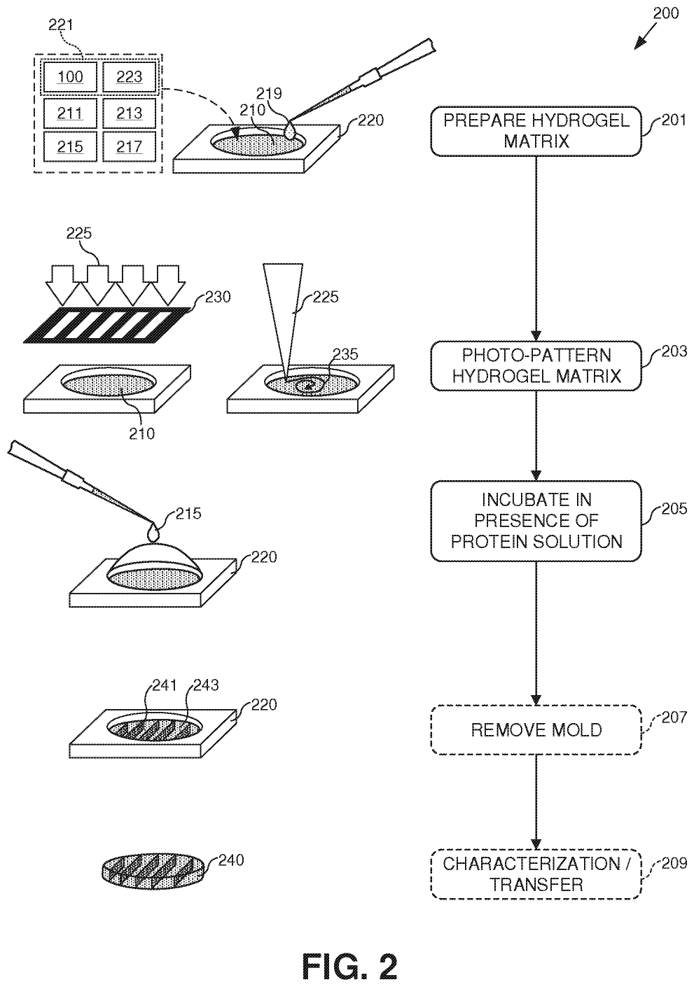

In some embodiments, example heterobifunctional linker 100 includes a peptide-reactive activated functional group 105 that can be or include, but is not limited to, an activated amine-reactive functional group, an activated thiol-reactive functional group, or any combination thereof, where the peptide-reactive activated functional group 105 is reactive with a hydrogel matrix 210 (in reference to FIG. 2A) that includes a crosslinked natural polymer. The peptide-reactive activated functional group 105 can be or include, but is not limited to, a N-hydroxysuccinimidide (NHS) activated amine-reactive functional group (e.g., a NHS ester); a N-hydroxysulfosuccinimido (NHSS) activated amine-reactive functional group (e.g., a NHSS ester), an isocyanate, an isothiocyanate, a maleimide-activated thiol-reactive functional group.

In some embodiments, example heterobifunctional linker 100 includes a photocaged reactive group 110 on the heterobifunctional linker 100 that includes a photocaged hydroxylamine, a photocaged alkoxyamine, a photocaged hydrazide, a photocaged amine, a photocaged tetrazine, or a photocaged alkyne-containing moiety (e.g., a photocaged cyclooctyne). To that end, the photocaged reactive group can be or include a photocage moiety 115 and a reactive group 120, where the reactive group 120 is exposed by radiation-mediated removal of the photocage 115.

The photocaged reactive group 110 can be adapted to be uncaged to expose the reactive group 120 following exposure to electromagnetic radiation (e.g., energetic photons) having an energy within a predetermined energy range. Photons having an energy within the predetermined energy range can be absorbed by the photocage 115 to provide a reactive hydroxylamine, alkoxyamine, a reactive hydrazide, a reactive tetrazine, a reactive amine, or a reactive alkyne (e.g., a reactive cyclooctyne), making up at least part of the reactive group 120. As described in more detail in reference to the forthcoming figures and examples, the energy range can be determined at least in part by the chemical structure of the caging group. In this way, the chemical structure of example heterobifunctional linker 100, and the energy range for uncaging photocage 115, can be selected for compatibility with living cells and also to facilitate photochemical uncaging in a manner orthogonal to the action of peptide-reactive group 105.

In some embodiments, the photocage 115 can be or include, but is not limited to, an optionally substituted 2-(2-nitrophenyl) propoxycarbonyl moiety, an optionally substituted nitrobenzyl moiety, an optionally substituted coumarin moiety, an optionally substituted BODIPY moiety, an optionally substituted ruthenium complex. Substituents can be or include, but are not limited to methyl, methoxy, carboxylic acid, ethyl, ethoxy, alkyl, aryl, —OH, —OR, —OC₆H₅, —NH₂, —NR₂, —NHCOCH₃, —CH₂Cl, —F, —Cl, —Br, —I, —CH═CHNO₂, —NO₃, —NH₃+, —PR₃+, —SR₂, —IC₆H₅, —CF₃, —CCl₃, —SO₃H, —SO₂R, —CO₂H, —CO₂R, —CONH₂, —CHO, —COR, —CN, or combinations thereof. In some embodiments, the combination of substituents affects the wavelength of electromagnetic radiation at which photocage 115 is removed to uncage reactive group 120. In this way, the range of energies over which heterobifunctional linker 100 is uncaged can be tailored to be cyto-compatible and to be accessible by an existing radiation source to be used for spatiotemporal functionalization. In an illustrative example, a hydrogel labeling kit can be prepared for a particular radiation source that operates at a wavelength from about 300 to about 500 nm, such as 365 nm, 463 nm, 468 nm, up to and including wavelengths in the visible and near-infrared ranges (from about 380 nm to about 900 nm) or the like. For example, in reference to metal-complex photocages 115, different ruthenium complexes can shift a peak wavelength from about 420 nm to about 500 nm, to about 600 nm, to about 770 nm, through substitution of pyridinic groups and introduction of carbonyl groups bound to the central ruthenium atom.

In some embodiments, the peptide-reactive activated functional group 105 does not include an azide. Azides form part of an established peptide ligation technique, referred to as Staudinger ligation. Azides are also photosensitive, however, over the same or similar photon energy ranges that can be used to uncage the photocaged reactive group 110. In this way, azide photochemistry can result in uncontrolled reaction between example heterobifunctional linker 100 with proteins and/or hydrogel 210 during exposure to electromagnetic radiation intended to uncage reactive group 120. For at least this reason, azides can impair the function of example heterobifunctional linker 100, at least in part by inducing uncontrolled photochemical reactions that interfere with 4D patterning of hydrogels 210. Additionally, azides offer relatively limited utility in labeling natural protein-based hydrogels, compared to heterobifunctional linker 100.

Example heterobifunctional linker 100 can be described by structures including, but not limited to those described by the following formulae:

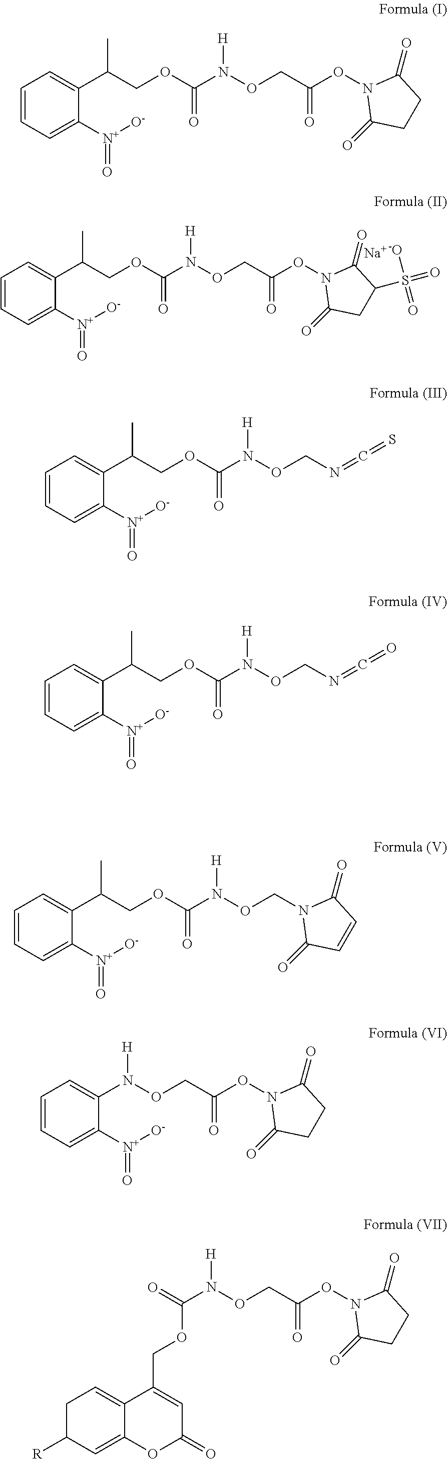

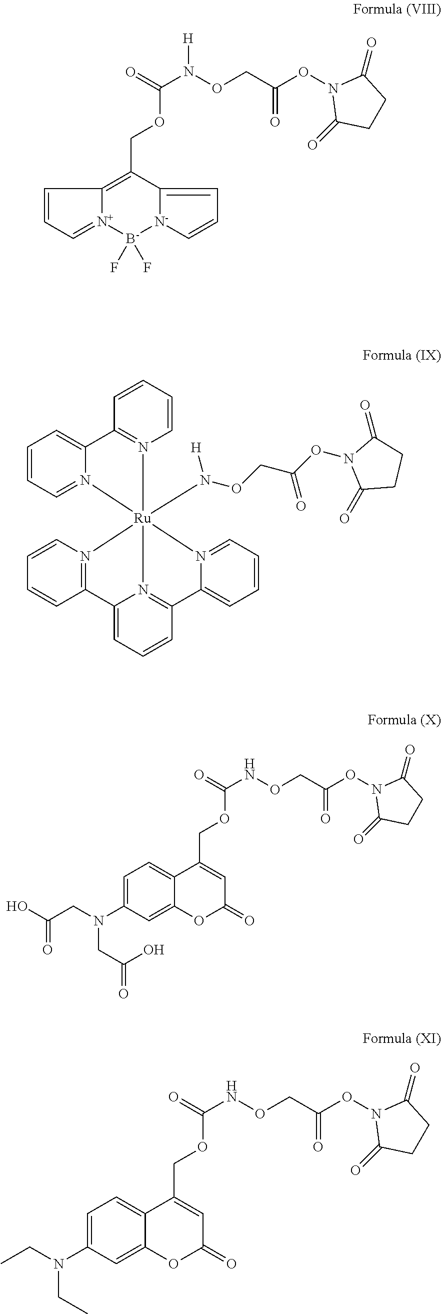

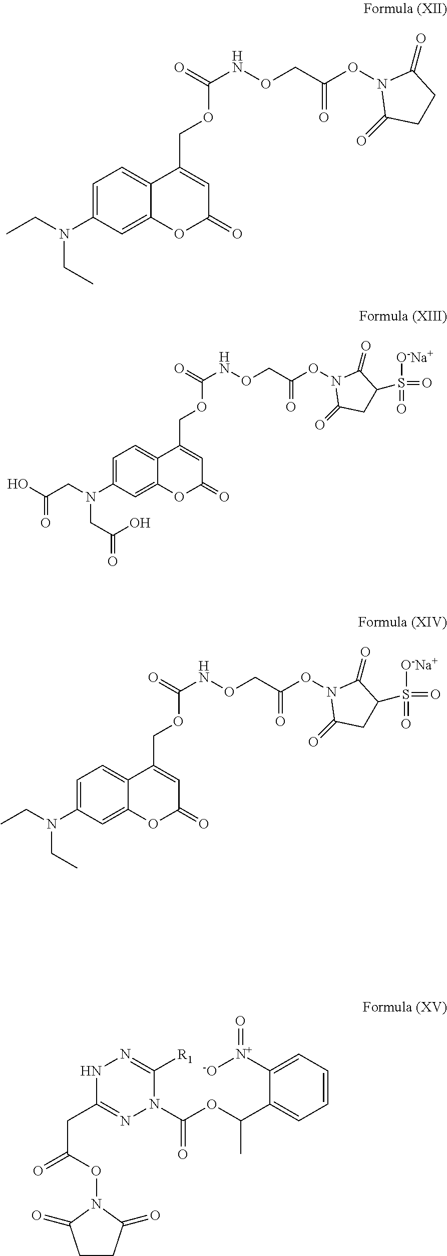

The chemical structure of photocage 115 influences the energy range over which reactive group 120 will be uncaged, based at least in part on the characteristic absorbance properties of photocage 115. In this way, photocage 115 can be selected from among the photo-active groups that absorb energetic photons in a cyto-compatible energy range, including but not limited to energies corresponding to wavelengths in the near-ultraviolet range from about 200 nm to about 400 nm, such as about 350 nm. In some embodiments, example heterobifunctional linker 100 can include a photocage 115 described by one of the following structural formulae:

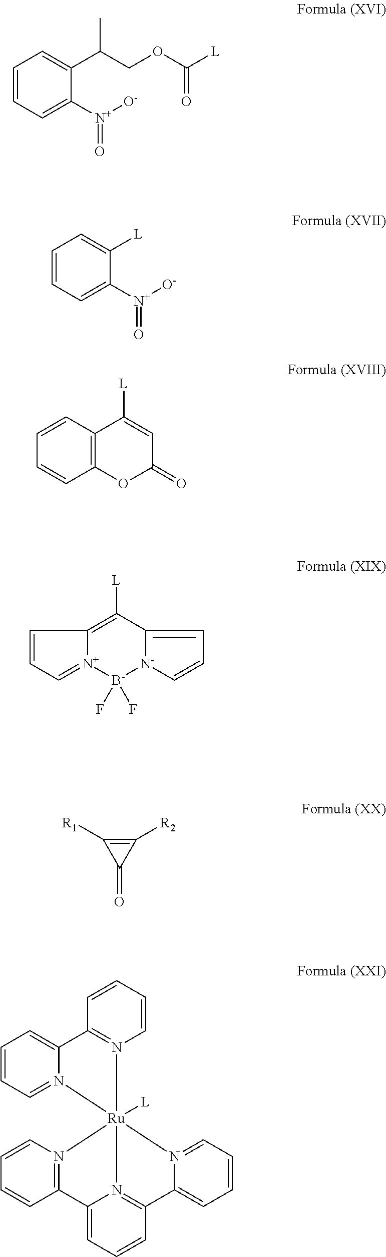

Formula (XIV) illustrates a single biologically active ligand (L) bound to a polypyridinic-ruthenium complex, which in this context represents reactive group 120. For metal coordination complexes, the chemical structure of the photocage ligands bound to the metal center strongly influences photo-chemical properties of the complex. In this way, alternative metals, photocage ligand groups, and/or structures can be included as a metal coordination complex to be used as photocage 115, which can be bound to one or more biologically active ligands (L).

The chemical structure of peptide reactive functional group 105 can influence the type of peptide to which example heterobifunctional linker can bind. For example, activated amine-reactive functional groups configure example heterobifunctional linker 100 to bind to amine moieties, while activated thiol-reactive functional groups configure example heterobifunctional linker 100 to bind to thiol moieties of hydrogel 210. In this way, example heterobifunctional linker 100 can be structured to covalently link to a hydrogel matrix including crosslinked natural polymers via a moiety including but not limited to —NHCO—, —C(O)O—, or —S—. In some embodiments, peptide reactive functional group 105 is described by one of the following structural formulae:

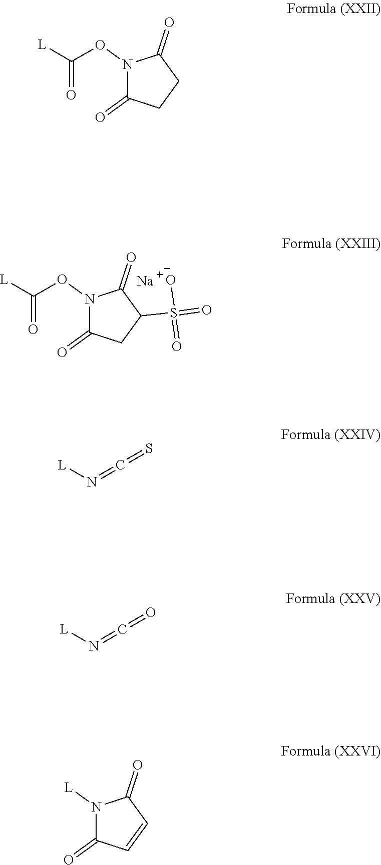

In some embodiments, reactive group 120 (L, above) can be described by one of the following structural formulae:

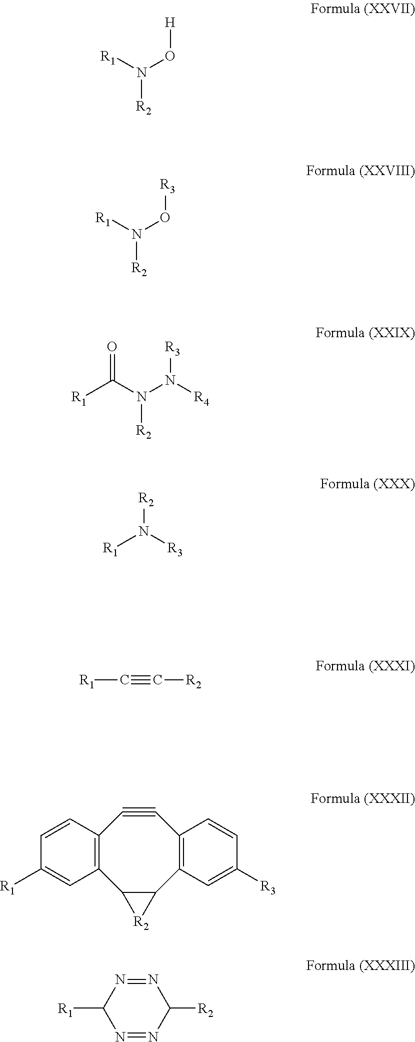

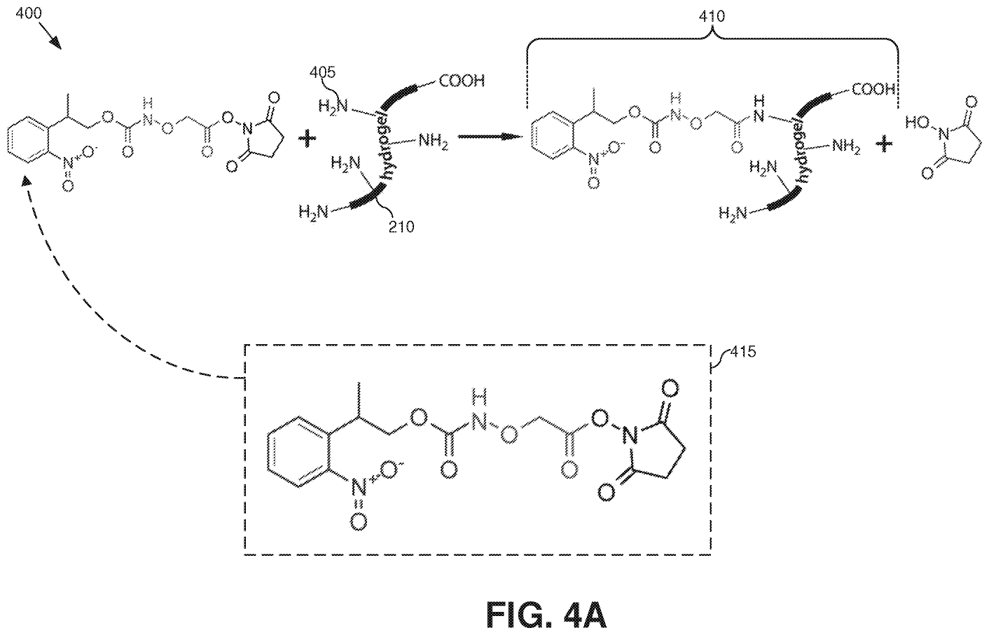

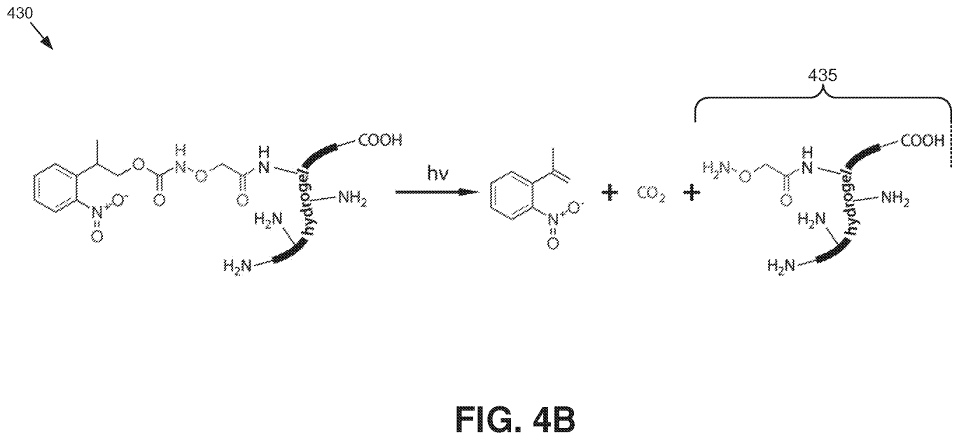

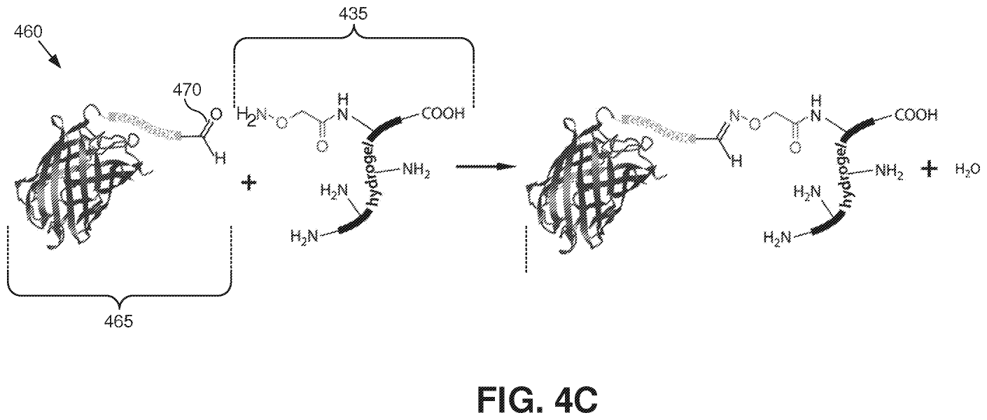

In the structural formulae (XXVII)-(XXXIII), above, R₁, R₂, R₃, and/or R₄ can independently represent photocage 115, peptide reactive group 105, or other moieties. For example. Formula (I) can be understood to be formula (XXVII) combined with formula (X) at R₂ and formula (XXII) at R₃, with R₁ being hydrogen. For Formula (XXXIII), a tetrazine moiety, photocage 115 can be linked at one of the nitrogen atoms, as illustrated in Formula (XV). In this way, example heterobifunctional linker 100 can be or include a molecule including two different reactive groups that are orthogonally activated by different physical mechanisms, permitting controlled ligation to two different biomolecules, as described in more detail in reference to FIGS. 4A-4C and Example 1.

FIG. 2 is a schematic diagram illustrating an example process 200 for preparing a patterned hydrogel 210 including a covalently linked hydrogel label 465 (in reference to FIG. 4C), in accordance with embodiments of the present disclosure. The operations of example process 200 illustrate process steps for implementing an orthogonal photo-mediated ligation reaction scheme that is described in more detail in reference to Example 1 and FIGS. 4A-4C. Example process 200 includes one or more optional operations that can be omitted or reordered and excludes one or more commonly known operations to simplify description of the method and to focus on operations for functionalization of hydrogel matrix 210.

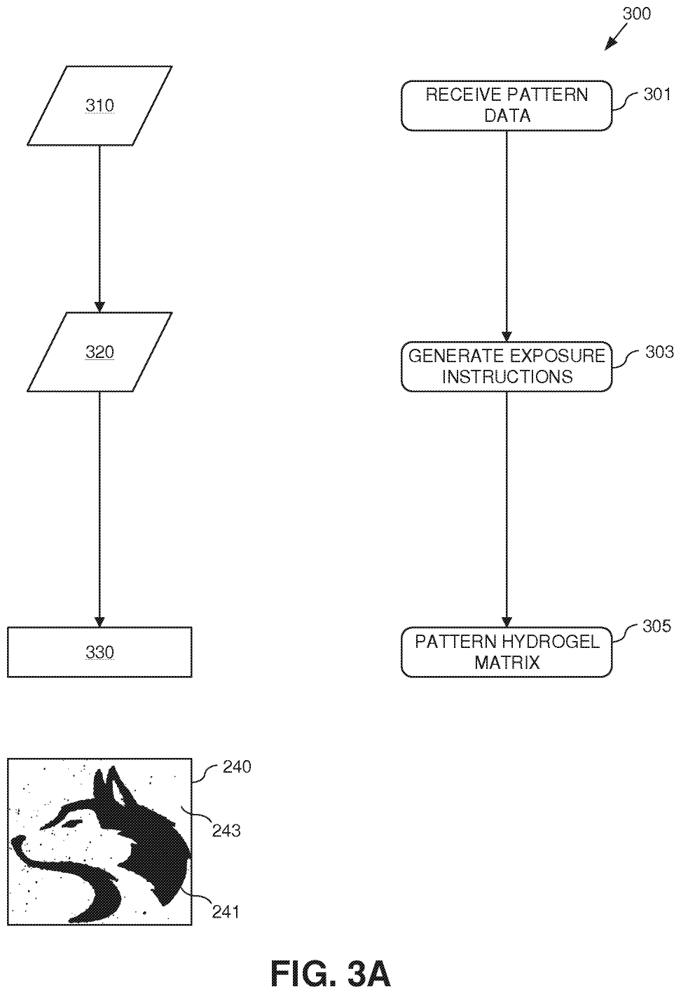

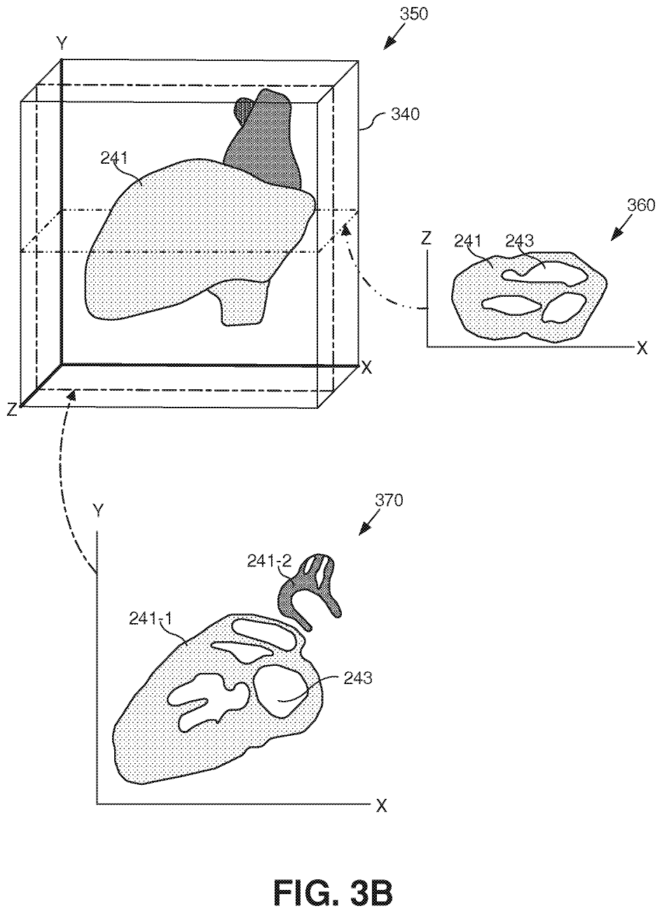

In some embodiments, example process 200 includes preparing hydrogel matrix 210 at operation 201. Preparing hydrogel matrix 210 can include operations for forming a hydrogel that includes one or more crosslinked natural polymer(s) 211. Crosslinking of polymer chains is responsible for forming the hydrophilic polymer network that later absorbs aqueous media in relatively large volumes up to and including 80% by weight. Preparation of synthetic hydrogels is typically undertaken by polymerization of a gel precursor (e.g., a monomer) in a reaction medium including initiator and cross-linker molecules. In contrast, natural polymer hydrogels can be prepared by crosslinking hydrophilic natural polymers rather than polymerizing (e.g., linear collagen, linear fibrin, etc.). In some cases, natural polymers can be functionalized with crosslinker target groups to facilitate crosslinking (e.g., in polysaccharide hydrogels). Additionally, operation 201 can include binding example heterobifunctional linker 100 to crosslinked natural polymer(s) 211, as described in more detail in reference to FIG. 3A. For example, binding can include selective reaction of peptide reactive group 105 with amine groups on crosslinked natural polymer(s) 211 to provide hydrogel matrix 210 with a substantially uniformly distribution of photocaged reactive groups 110. Amine groups can include N-terminus groups and/or amine side chains 405 (in reference to FIG. 4A), as described in more detail in reference to FIG. 4A-4C.

It is understood that operation 201 can include one or more sub-operations, such as curing, medium exchange, atmosphere exchange, degassing, or the like, that would be understood by a person having ordinary skill in the chemical arts. In an illustrative example, operation 201 includes casting hydrogel matrix 210 by depositing a liquid gel precursor 219 in a mold 220, where liquid gel precursor 219 can be or include a suspension of non-crosslinked natural polymers. In addition, hydrogel matrix 210 can include living cells 213, heterobifunctional linker 100, hydrogel label(s) 215, and/or cell media 217. In this way, hydrogel matrix 210 can include living cells 213 and cell media 217 to maintain vitality of living cells 213. An example method for casting hydrogel matrix 210 is provided in Example 1, below.

In some embodiments, preparing hydrogel matrix 210 can include additional operations of activating hydrogel label(s) 215 using a hydrogel label activator 223. As described in more detail in reference to FIGS. 4A-C, hydrogel label(s) 215 can be or include a protein or other biomolecule that is conjugated with or otherwise includes a reactive group 470 (in reference to Example 4C) that can react with reactive group 120 to bind crosslinked natural polymers 211 with hydrogel label(s) 215. In some embodiments, hydrogel label activator 223 reacts with and activates reactive group 475 on hydrogel label(s) 215 (e.g., conjugates an aromatic aldehyde to a protein). Reactive group 475 on hydrogel label(s) 215 can be or include, but is not limited to, a carbonyl containing group, such as an aldehyde, a carboxylic acid, or an ester. In some embodiments, hydrogel label activator 223 is structured to tether azides and trans-cyclooctenes to proteins, for example, through OSu or an activated ester similar to structures illustrated in Formulae (XXII-XXVI) in reference to peptide reactive functional group 105, as would be understood by a person having ordinary skill in the chemical arts. In an illustrative example, hydrogel label activator 223 can be or include a reagent for providing an carbonyl group on hydrogel label(s) 215 based on Formula XXXIV. For example, hydrogel label activator 223 can be or include 2-5-dioxopyrrolidin-1-yl 4-formylbenzoate and its functional equivalents, illustrated below:

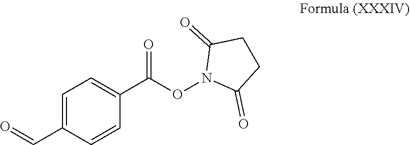 [SMILES: O=Cc1ccc(C(=O)ON2C(=O)CCC2=O)cc1]

In this way, reactive group 120, as described in more detail in reference to FIG. 1, can be selected in coordination with the hydrogel label(s) 215 and the hydrogel label activator 223 to introduce multi-step control of reaction processes including activation of hydrogel label(s) 215 and uncaging of reactive group 120. For example, reactive group 475 on hydrogel label 215 can be adapted to react with reactive group 120 of photocaged reactive group 100 of example heterobifunctional linker 100, when uncaged.

In some embodiments, example heterobifunctional linker 100 is provided with hydrogel label activator 223 as part of a hydrogel-labeling kit 221. Hydrogel-labeling kit 221 can include example heterobifunctional linker 100 and hydrogel-label activator 223 dissolved in a liquid medium, for example, in an aqueous medium including, but not limited to water, cell media, or the like. Hydrogel-labeling kit 221 can include separate containers for each component or can include each component in a single container. To reduce unintended photo-chemical reaction during storage and transport of hydrogel labelling kit 221, at least example heterobifunctional linker 100 can be stored in a container that is opaque to electromagnetic radiation over an energy range including the energy used to uncage the photocage 115. Hydrogel-labeling kit 221 can be stored in a transparent plastic container inside an opaque sleave. To limit spontaneous reaction, hydrogel labelling kit 221 can be stored at or below its freezing temperature until ready for use.

In some embodiments, example process 200 includes photo-patterning hydrogel matrix 210 at operation 203. Photo-patterning hydrogel matrix can include selectively exposing hydrogel matrix 210 to electromagnetic radiation 225 having an energy within a predetermined energy range to uncage photocage 115 and expose reactive group 120. Selective exposure can include spatiotemporal patterning of hydrogel matrix 210 in two or three-dimensions. In an example, photo-patterning can be implemented as a function of time, both by exposing hydrogel matrix 210 at a predetermined time and for a predetermined duration. In another example, photo-patterning can be implemented as a function of position in hydrogel matrix 210 at one or more predetermined locations. In this way, reactive group 120 can be uncaged at the predetermined time and the predetermined location, in accordance with the localization of the exposure.

As part of operation 203, photo-patterning selectively activates reactive group 120 of heterobifunctional linker 100 to enable binding with hydrogel label 215 (e.g., activated hydrogel label 215) in a spatiotemporally controlled manner. In some embodiments, the extent of binding can be controlled in advance by duration and intensity of exposure, which can be described as a radiation “dose.” Photo-patterning can include pulsed exposure using a radiation source (e.g., a near-UV laser or LED source) that operates by a duty-cycle, such that the total exposure time and the total dose can be independently controlled. Advantageously, “dosing” radiation 225 in this way can form a pattern 235 in hydrogel matrix 210 in a cyto-compatible manner, for example, by selectively activating reactive group 120 in a spatially localized manner while also maintaining viability of cells 213. In this way, cell migration, proliferation, and other cellular processes can be guided in 4D through the formation of pattern(s) 235 of bound example heterobifunctional linker 100, examples of which are described in reference to Examples 1-10, below.

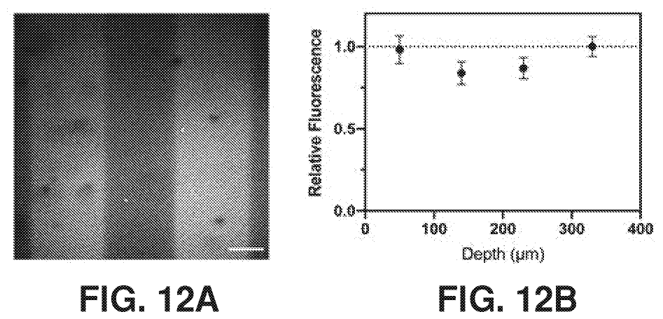

In some embodiments, photo-patterning can include exposing hydrogel matrix 210 to electromagnetic radiation 225 through a mask 230 using a uniform source (e.g., a collimated LED panel or a parallel source), to transfer a two-dimensional pattern into hydrogel matrix 210. Since absorbance of radiation 225 in hydrogel matrix 210 can occur along a path through the hydrogel medium (e.g., described by the Beer-Lambert law or its variations), masking can impart a three-dimensional pattern in hydrogel matrix 210 that can be pre-determined through simulation or otherwise determined for the hydrogel material, as described in more detail in reference to FIGS. 12A-12B. In some embodiments, a focused source of radiation 225 can be applied to transfer an arbitrary pattern 235 into hydrogel matrix 210 (e.g., using laser-scanning lithography), as described in more detail in reference to FIG. 3A and FIG. 3B.

In some embodiments, example process 200 includes reacting the uncaged reactive group 120 with hydrogel label 215 at operation 205. Reacting reactive group 120 with hydrogel label 215 can include incubating hydrogel matrix 210 in the presence of hydrogel label 215 at a given temperature and for a given time, such that the hydrogel label 215 binds to reactive group 120 to produce a patterned hydrogel 240. In this way, uncaged reactive group 120 can be understood to undergo a bio-orthogonal reaction with hydrogel label 215.

In some embodiments, hydrogel label 215 includes a complementary reactive group configured to react with the uncaged reactive group 120 of the example heterobifunctional linker 100 (e.g., an aldehyde). In some embodiments, example process 200 can optionally include disposing hydrogel label 215 in or onto hydrogel matrix 210 as part of operation 205. For example, introducing hydrogel label 215 after exposure to radiation 225 can be indicated where hydrogel label 215 is sensitive or otherwise absorbs radiation 225 in a way that would otherwise interfere with the formation of patterned hydrogel 240. In the illustrated embodiment, patterned hydrogel 240 includes labeled regions 241 and unlabeled regions 243, corresponding to pattern 235 transferred by mask 230.

In some embodiments, example process 200 optionally includes operations 207 and 209 to isolate patterned hydrogel 240 from mold 220 for characterization of protein function, cell vitality and proliferation, and transfer of patterned hydrogel 240 into a host or different medium. In some embodiments, transfer can include implantation of patterned hydrogel 240 in a living host, such as a model organism (e.g., mouse model, rat model, pig model, etc.), for example, for perfusion or other in vivo processes. In some embodiments, patterned hydrogel 240 can be designed to serve as a scaffold for proliferation and migration of native cells in an implant host, for example, to improve wound healing or nerve regeneration. In this way, operations 107-109 can facilitate the application of patterned hydrogel 240 in experimental medicine, therapeutics development, regenerative medicine, or other fields. For example patterned hydrogel 240 can be patterned with an array of different microenvironments corresponding to different radiation doses.

FIG. 3A is a schematic diagram illustrating operations of the example process 300 for preparing a patterned hydrogel with an arbitrary pattern 235 formed in two or three spatial dimensions and in a temporal dimension, in accordance with embodiments of the present disclosure. Example process 300 is an illustrative embodiment of example process 200, including additional optional operations for converting arbitrary pattern data 310 into pattern 235 in patterned hydrogel 240. As described in more detail in reference to FIG. 2, pattern 235 can be formed by transferring a pattern from mask 230 or by localized irradiation in three dimensions. The operations of example process 300 can be implemented automatically (e.g., without human intervention) by a computer system in electronic communication with a radiation source (e.g., a laser or LED source) configured to emit radiation in a predetermined energy range selected to uncage photocaged reactive group 110. In this way, the individual operations of example process 300 can be incorporated into a manufacturing process or other parallelized system for the production of hydrogel matrices bearing arbitrary patterns 235 and, in some cases, living cells 213. In some embodiments, example process 300 is implemented at a separate location from at least some operations of example process 200, such that preparation of hydrogel matrix, seeding with living cells 213, and providing hydrogel labeling kit 221 can be completed in advance, for example, at a facility that prepares, packages, and ships hydrogels for therapeutic screenings or other medical uses.

In some embodiments, example process 300 includes receiving pattern data 310 at operation 301. Pattern data can be computer-readable numerical data, image data, exposure data, or the like, that the computer system can receive from a user device or via a network. In some embodiments, pattern data can be received as a color image, where each color encodes a quantized dose of electromagnetic radiation. In some embodiments, pattern data can include a greyscale image, where the alpha value can encode an incremental dose between a maximum dose (e.g., k=1) and a minimum dose (e.g., k=0).

In some embodiments, example process 300 includes generating exposure instructions 320 using pattern data 310. Generating exposure instructions 320 can include processing pattern data 310 to map intensity, location, and/or timing data into spatiotemporal control instructions to control a radiation source including a moveable stage for hydrogel matrix 210 and a source of electromagnetic radiation. In an illustrative example, the radiation source is a laser-scanning lithographic platform configured with one or more steerable lasers configured to irradiate precise volumes of hydrogel matrix 210 with corresponding doses of UV radiation, in accordance with pattern data 310. In another illustrative example, the radiation source includes a translation/rotation stage with multiple degrees of freedom (e.g., a three-axis translation stage, a six degree-of-freedom stage, or the like) that can be moved relative to a fixed or movable source of electromagnetic radiation (e.g., a laser or LED source) in the predetermined wavelength to uncage reactive group 120 of example heterobifunctional linker 100. In this way, exposure instructions 320 can include coordinated movement instructions for the stage of the radiation source and power/output instructions for the source of electromagnetic radiation that together define the spatiotemporally localized dosages of electromagnetic radiation at one or more locations of hydrogel matrix 210. In another illustrative example, the radiation source includes a movable stage and multiple sources of electromagnetic radiation that individually provide doses low enough to affect a limited uncaging of reactive group 120 of example heterobifunctional linker 100, but can be focused to a common volume of hydrogel matrix from different angles to uncage reactive group 120 within the common volume, but not outside the common volume. In this way, exposure instructions 320 can include movement instructions for the moveable stage that are coordinated with movement and/or timing instructions for the multiple sources of electromagnetic radiation. In another illustrative example, radiation source includes an addressable filter array of addressable pixels (e.g., liquid crystal elements, addressable color filters, etc.) that can be reversibly switched between a transmissive state and a non-transmissive state that can be used to define mask 230. In this way, exposure instructions can include filter instructions corresponding to a binary mask image generated from pattern data 310 (e.g., by intensity thresholding) and timing/power instructions for a planar source of electromagnetic radiation. In another illustrative example, pattern data can be represented as a physical mask 230, such as a polymer film or other material that selectively absorbs or otherwise blocks photons having energy in the range for uncaging reactive group 120.

As would be understood by a person having ordinary skill in the optical arts, additional and/or alternative techniques may be applied to pattern hydrogel matrix 210 using electromagnetic radiation beyond the illustrative examples described herein, including but not limited to fixed radiation sources with moveable optics, addressable LED arrays with collimating optics to provide two-dimensional exposure patterns analogous to mask 230, or the like.

In some embodiments, example process 300 includes patterning hydrogel matrix 210 at operation 305. With respect to an automated system including a moveable stage and a source of electromagnetic radiation, operation 305 can include controlling (e.g., automatically) the moveable stage such that hydrogel matrix 210 is translated and/or rotated in two or three-dimensions according to a set of instructions that permits a particular dose of electromagnetic radiation to be delivered to one or more volumes of hydrogel matrix 210 to produce patterned hydrogel 240. In an illustrative example, moveable stage can be a three-axis translation stage and source of electromagnetic radiation can include a first fixed laser source aligned with a vertical “z” axis of the translation stage and a second fixed laser source aligned with a horizontal “x” or “y” axis of the translation stage, where the first fixed laser source and the second fixed laser source intersect. In this way, operation 305 can include translating hydrogel matrix 210 in two horizontal dimensions over a series of discrete z-planes while coordinating different total dosage at the intersection of the laser sources with the position of the stage to define a three-dimensional pattern 235. As would be understood by a person having ordinary skill in the optical arts, two and three-dimensional exposure patterns can be effected in multiple different stage and source configurations including, but not limited to moving a stage relative to a fixed source, steering a beam relative to a fixed or moveable stage, or the like.

FIG. 3B is a schematic diagram illustrating an example patterned hydrogel 340 in three spatial dimensions prepared by the example processes 200 and/or 300 of FIGS. 2-3A, in accordance with embodiments of the present disclosure. FIG. 3B illustrates a simulated multichambered heart in a perspective view 350, an x-z section view 360, and an x-y section view 370, where labeled regions 241 reproduce the shape of heart tissue and unlabeled regions 243 reproduce internal volumes and the region of hydrogel matrix 210 external to labeled regions 241. As illustrated in section views 360 and 370, example patterned hydrogel 340 includes multiple hidden geometries formed by selectively exposing regions of hydrogel matrix 210 with electromagnetic radiation having an energy within a range to uncage reactive group 120 of heterobifunctional linker 100 and subsequently binding uncaged reactive group 120 with hydrogel label(s) 215 to define labeled regions 241. The resulting patterned hydrogel 241 includes hydrogel matrix 210 including crosslinked natural polymer(s) 211 and covalently linked hydrogel label(s) 215 at predetermined locations in hydrogel matrix 210, wherein hydrogel label(s) 215 can be covalently linked to the hydrogel matrix 210 via example heterobifunctional linker 100.

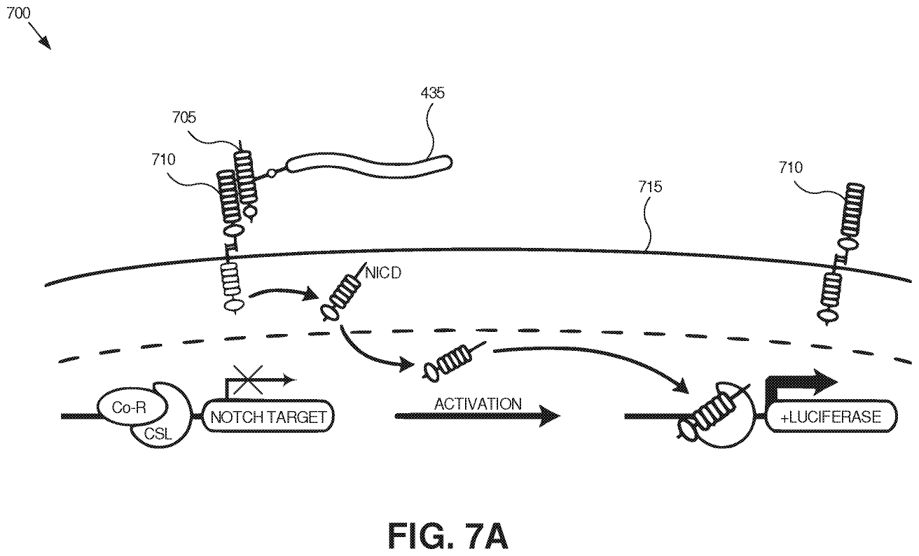

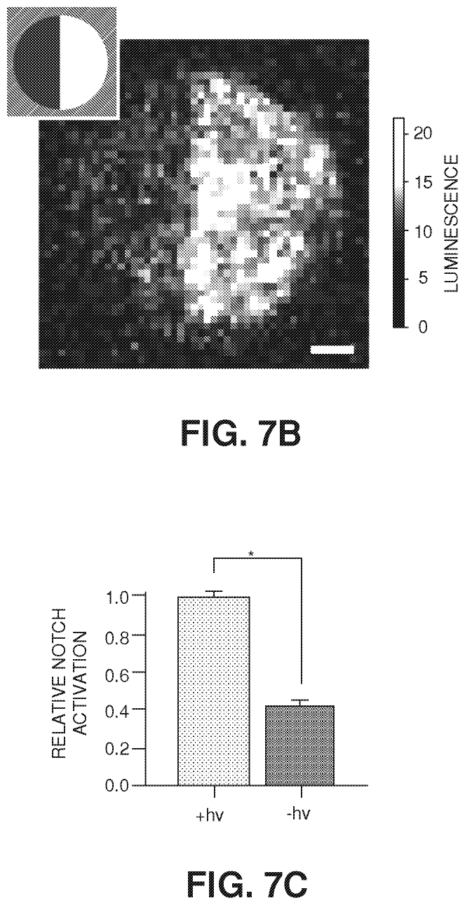

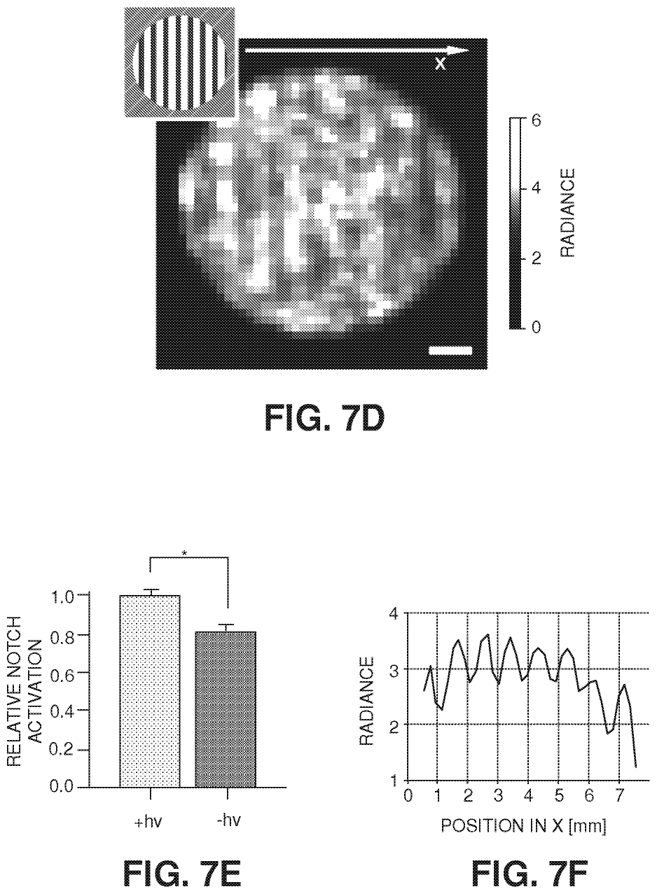

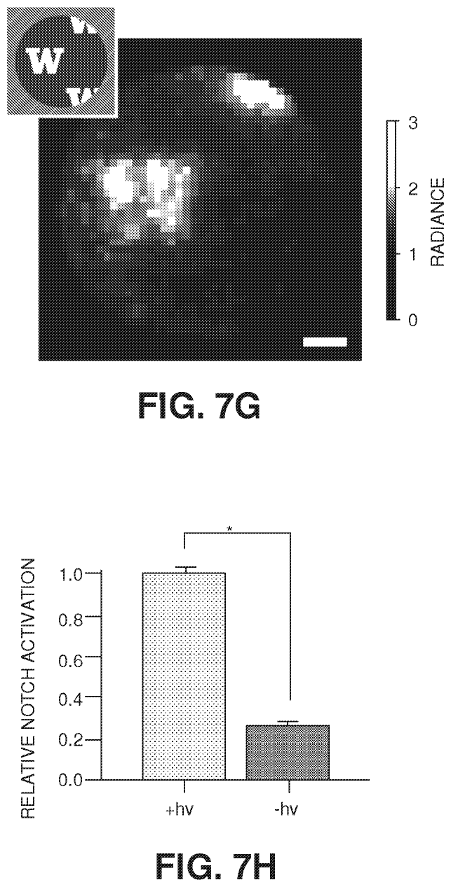

In some embodiments, crosslinked natural polymer can be selected from crosslinked fibrin, crosslinked collagen, crosslinked gelatin, and crosslinked decellularized extracellular matrix. In some embodiments, hydrogel label(s) 215 are selected from a peptide, a polypeptide, a saccharide, a polysaccharide, a fluorescent molecule, and a radio-labeled molecule. While illustrated as a smooth and continuous three-dimensional volume, labeled regions can also be regularly spaced in hydrogel matrix 210. In this way, multiple labeled micro-environments can be defined in hydrogel matrix 210 that are fluidically coupled with each other for diffusion of small molecules and/or living cells 213, but can otherwise implement different conditions between the micro-environments. In an illustrative example, varying dosage of electromagnetic radiation can modulate the extent of label binding between the micro-environments. As described in more detail in reference to FIGS. 7A-7H, different spatially separate environments can be used to regulate microbiological processes as part of multi-parameter screening experiments, cell seeding and proliferation, therapeutic screening with internal control, or the like.

In some embodiments, patterned hydrogel 340 includes multiple different labeled regions 241, including different labels or different spatial or temporal patterns. For example, example patterned hydrogel 340 can include a first labeled region 241-1 that is characterized by a first set of properties and a second labeled region 241-2 that is characterized by a second set of properties, as shown in section view 370. In this example, first labeled region 241-1 corresponds to the heart muscle and second labeled region 241-2 corresponds to a region of an artery coupled with the heart muscle. To provide physiologically meaningful interaction between labeled regions 240 and living cells 213, first labeled region 241-1 and second labeled region 241-2 can have different properties including geometric properties of labeling, including metapatterning within labeled region 241-2, different densities of labeling (e.g., by varying dosage of electromagnetic radiation), different labels (e.g., where hydrogel matrix 210 is prepared with multiple different labels 215 paired with multiple different photocaged reactive groups 110 on multiple different linkers 100 that are activated by different energies). In an illustrative example, living cells 213 can be induced to self-organize in different ways in different labeled regions 214 to correspond to typical orientations in native tissues. For example, arteries can include cells organized relative to a central flow axis (e.g., radial alignment or longitudinal alignment) of the artery, while the heart itself can include cells that are arranged relative to chambers of the heart, represented as unlabeled regions 243. Similarly, a patterned hydrogel 340 that models a liver tissue can include different regions to correspond to different cellular environments of the organ. Advantageously, patterned hydrogel 340 can simulate, in vitro, a model tissue by reproducing the environment of an organ or a portion of an organ.

### Definitions

At various places in the present specification, substituents of compounds of the disclosure are disclosed in groups or in ranges. It is specifically intended that the disclosure include each and every individual subcombination of the members of such groups and ranges. For example, the term “C1-6 alkyl” is specifically intended to individually disclose methyl, ethyl, C3 alkyl, C4 alkyl, C5 alkyl, and C6 alkyl.

It is further appreciated that certain features of the disclosure, which are, for clarity, described in the context of separate embodiments, can also be provided in combination in a single embodiment.

Conversely, various features of the disclosure which are, for brevity, described in the context of a single embodiment, can also be provided separately or in any suitable subcombination.

As used herein, the term “substituted” or “substitution” refers to the replacing of a hydrogen atom with a substituent other than H. For example, an “N-substituted piperidin-4-yl” refers to replacement of the H atom from the NH of the piperidinyl with a non-hydrogen substituent such as, for example, alkyl.

As used herein, the term “activating agent” refers to a chemical compound which is capable of activating, for example, one or more carboxyl groups within carboxylic acids or carboxylic acid derivatives for nucleophilic reactions, wherein preferably said carboxyl groups include —C(O)X groups, wherein X=OH, halo (e.g., I, Br, Cl), OR (e.g., an anhydride), NH2 or NH—R. In some embodiments, the carbonyl group is within a chloroformate. An “activated” functional group is a functional group that has been reacted with an activating agent. The activated functional group has a lower barrier to reacting with a nucleophile compared to an unactivated functional group.

As used herein, the term “natural polymer,” “naturally derived polymer,” or “naturally sourced polymer” refers to polymers found in nature. Examples of natural polymers include polynucleotides, polypeptides, and polysaccharides. Examples of polypeptides include but are not limited, to collagen, fibrin, gelatin, decellularized extracellular matrix, Matrigel, and actin (e.g., F-actin).

As used herein, the term “caging group” or “caging” refers to a moiety that can be employed to reversibly block, inhibit, or interfere with the activity (e.g., the chemical reactivity) of a molecule (e.g., a polypeptide, a nucleic acid, a small molecule, a drug, and the like) and its respective process. Typically, one or more caging groups are associated (covalently or noncovalently) with the molecule but do not necessarily surround the molecule in a physical cage. Caging groups can be removed from a molecule, or their interference with the molecule's activity can be otherwise reversed or reduced, by exposure to an appropriate type of uncaging energy and/or exposure to an uncaging chemical, enzyme, or the like. Examples of caging groups that can be used in the heterobifunctional linker are described, for example, in Dynamic Studies in Biology: Phototriggers, Photoswitches, and Caged Biomolecules Edited by Maurice Goeldner (Universitd L. Pasteur Strasbourg, France) and Richard Givens (University of Kansas, USA). Wiley-VCH GmbH & Co. KGaA: Weinheim. 2005, incorporated herein by reference in its entirety.

As used herein, the term “photocaging” refers to a caging group that is removed by exposing the caging group to photons having an energy within a predetermined energy range. The energy range can be determined at least in part by the chemical structure of the caging group, such that the energy range can be selected for compatibility with living cells and also to facilitate photo-chemical removal in an orthogonal manner to a second, different, chemical linking modality.

As used herein, the term “linker” refers to atoms or molecules that link or bond two entities (e.g., hydrogel, hydrogel label, solid supports, oligonucleotides, or other molecules), but that is not a part of either of the individual linked entities.

As used herein, the term “alkyl” refers to a saturated hydrocarbon group which is straight-chained (e.g., linear) or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 30, from 1 to about 24, from 2 to about 24, from 1 to about 20, from 2 to about 20, from 1 to about 10, from 1 to about 8, from 1 to about 6, from 1 to about 4, or from 1 to about 3 carbon atoms.

As used herein, the term “aryl” refers to monocyclic or polycyclic (e.g., having 2, 3, or 4 fused rings) aromatic hydrocarbons such as, for example, phenyl, naphthyl, anthracenyl, phenanthrenyl, indanyl, and indenyl. In some embodiments, aryl groups have from 6 to about 20 carbon atoms.

As used herein, the term “halo” or “halogen” includes fluoro, chloro, bromo, and iodo.

As used herein, the term “alkylene” refers to a linking alkyl group.

As used herein, “alkenyl” refers to an alkyl group having one or more double carbon-carbon bonds. The alkenyl group can be linear or branched. Example alkenyl groups include ethenyl, propenyl, and the like. An alkenyl group can contain from 2 to about 30, from 2 to about 24, from 2 to about 20, from 2 to about 10, from 2 to about 8, from 2 to about 6, or from 2 to about 4 carbon atoms.

As used herein, “alkenylene” refers to a linking alkenyl group.

As used herein, “alkynyl” refers to an alkyl group having one or more triple carbon-carbon bonds. The alkynyl group can be linear or branched. Example alkynyl groups include ethynyl, propynyl, and the like. An alkynyl group can contain from 2 to about 30, from 2 to about 24, from 2 to about 20, from 2 to about 10, from 2 to about 8, from 2 to about 6, or from 2 to about 4 carbon atoms.

As used herein, “alkynylene” refers to a linking alkynyl group.

As used herein, “azide” refers to a group having one or more triple-nitrogen (N₃) groups.

As used herein, the term “random copolymer” is a copolymer having an uncontrolled mixture of two or more constitutional units. The distribution of the constitutional units throughout a polymer backbone can be a statistical distribution, or approach a statistical distribution, of the constitutional units. In some embodiments, the distribution of one or more of the constitutional units is favored. For a polymer made via a controlled polymerization (e.g., RAFT, ATRP, ionic polymerization), a gradient can occur in the polymer chain, where the beginning of the polymer chain (in the direction of growth) can be relatively rich in a constitutional unit formed from a more reactive monomer w % bile the later part of the polymer can be relatively rich in a constitutional unit formed from a less reactive monomer, as the more reactive monomer is depleted. To decrease differences in distribution of the constitutional units, comonomers in the same family (e.g., methacrylate-methacrylate, acrylamide-acrylamido) can be used in the polymerization process, such that the monomer reactivity ratios are similar.

As used herein, the term “constitutional unit” of a polymer refers to an atom or group of atoms in a polymer, comprising a part of the chain together with its pendant atoms or groups of atoms, if any. The constitutional unit can refer to a repeat unit. The constitutional unit can also refer to an end group on a polymer chain. For example, the constitutional unit of polyethylene glycol can be —CH₂CH₂O— corresponding to a repeat unit, or —CH₂CH₂OH corresponding to an end group.

As used herein, the term “repeat unit” corresponds to the smallest constitutional unit, the repetition of which constitutes a regular macromolecule (or oligomer molecule or block).

As used herein, the term “end group” refers to a constitutional unit with only one attachment to a polymer chain, located at the end of a polymer. For example, the end group can be derived from a monomer unit at the end of the polymer, once the monomer unit has been polymerized. As another example, the end group can be a part of a chain transfer agent or initiating agent that was used to synthesize the polymer.

As used herein, the term “terminus” of a polymer refers to a constitutional unit of the polymer that is positioned at the end of a polymer backbone.

As used herein, the term “biodegradable” refers to a process that degrades a material via hydrolysis and/or a catalytic degradation process, such as enzyme-mediated hydrolysis and/or oxidation. For example, polymer side chains can be cleaved from the polymer backbone via either hydrolysis or a catalytic process (e.g., enzyme-mediated hydrolysis and/or oxidation).

As used herein, “biocompatible” refers to a property of a molecule characterized by it, or its in vivo degradation products, being not, or at least minimally and/or reparably, injurious to living tissue; and/or not, or at least minimally and controllably, causing an immunological reaction in living tissue. As used herein, “physiologically acceptable” is interchangeable with biocompatible.

As used herein, the term “hydrophobic” refers to a moiety that is not attracted to water with significant apolar surface area at physiological pH and/or salt conditions. This phase separation can be observed via a combination of dynamic light scattering and aqueous NMR measurements. Hydrophobic constitutional units tend to be non-polar in aqueous conditions. Examples of hydrophobic moieties include alkyl groups, aryl groups, etc.

As used herein, the term “hydrophilic” refers to a moiety that is attracted to and tends to be dissolved by water. The hydrophilic moiety is miscible with an aqueous phase. Hydrophilic constitutional units can be polar and/or ionizable in aqueous conditions. Hydrophilic constitutional units can be ionizable under aqueous conditions and/or contain polar functional groups such as amides, hydroxyl groups, or ethylene glycol residues. Examples of hydrophilic moieties include carboxylic acid groups, amino groups, hydroxyl groups, etc.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, the term “anionic” refers to a functional group that is negatively charged, or ionizable to a negatively charged moiety under physiological conditions. Examples of anionic groups include carboxylate, sulfate, sulfonate, phosphate, etc.

As used herein, the term “peptide” refers to natural biological or artificially manufactured short chains of amino acid monomers linked by peptide (amide) bonds. As used herein, a peptide has at least 2 amino acid repeating units.

As used herein, the term “oligomer” refers to a macromolecule having 10 or less repeating units.

As used herein, the term “polymer” refers to a macromolecule having more than 10 repeating units.

As used herein, the term “polysaccharide” refers to a carbohydrate that can be decomposed by hydrolysis into two or more molecules of monosaccharides.

As used herein, the term “hydrogel” refers to a water-swollen, and cross-linked polymeric network produced by the reaction of one or more monomers. The polymeric material exhibits the ability to swell and retain a significant fraction of water within its structure, but does not dissolve in water.

As used herein, the term “protein” refers to any of various naturally occurring substances that consist of amino-acid residues joined by peptide bonds, contain the elements carbon, hydrogen, nitrogen, oxygen, usually sulfur, and occasionally other elements (such as phosphorus or iron), and include many essential biological compounds (such as enzymes, hormones, or antibodies).

As used herein, the term “tissue” refers to an aggregate of similar cells and cell products forming a definite kind of structural material with a specific function, in a multicellular organism.

As used herein, the term “organs” refers to a group of tissues in a living organism that have been adapted to perform a specific function.

As used herein, the term “therapeutic agent” refers to a substance capable of producing a curative effect in a disease state.

As used herein, the term “small molecule” refers to a low molecular weight (<2000 daltons) organic compound that may help regulate a biological process, with a size on the order of 1 nm. Most drugs are small molecules.

As used herein, the term “biomaterial” refers to a natural or synthetic material (such as a metal or polymer) that is suitable for introduction into living tissue, for example, as part of a medical device (such as an artificial joint).

As used herein, the term “ceramic” refers to an inorganic, non-metallic, solid material comprising metal, non-metal or metalloid atoms primarily held in ionic and covalent bonds.

As used herein, the term “composite” refers to a composition material, a material made from two or more constituent materials with significantly different physical or chemical properties that, when combined, produce a material with characteristics different from the individual components. The individual components remain separate and distinct within the finished structure.

As used herein, the term “chelating agent” refers to a ligand that forms two or more separate coordinate bonds to a single central metal ion.

One letter codes for amino acids are used herein. For example, alanine is A, arginine is R, asparagine is N, aspartic acid is D, asparagine or aspartic acid is B, cysteine is C, glutamic acid is E, glutamine is Q, glutamine or glutamic acid is Z, glycine is G, histidine is H, isoleucine is I, leucine is L, lysine is K, methionine is M, phenylalanine is F, proline is P, serine is S, threonine is T, tryptophan is W, tyrosine is Y, valine is V.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of a therapeutic agent (i.e., drug, or therapeutic agent composition) that elicits the biological or medicinal response that is being sought in a tissue, system, animal, individual or human by a researcher, veterinarian, medical doctor or other clinician, which includes one or more of the following:
(1) preventing the disease; for example, preventing a disease, condition or disorder in an individual who may be predisposed to the disease, condition or disorder but does not yet experience or display the pathology or symptomatology of the disease;
(2) inhibiting the disease; for example, inhibiting a disease, condition or disorder in an individual who is experiencing or displaying the pathology or symptomatology of the disease, condition or disorder; and
(3) ameliorating the disease: for example, ameliorating a disease, condition or disorder in an individual who is experiencing or displaying the pathology or symptomatology of the disease, condition or disorder (i.e., reversing the pathology and/or symptomatology) such as decreasing the severity of disease.

The compounds described herein can be asymmetric (e.g., having one or more stereocenters).

All stereoisomers, such as enantiomers and diastereomers, are intended unless otherwise indicated.

Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and can be isolated as a mixture of isomers or as separated isomeric forms.

Compounds of the disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the disclosure can also include all isotopes of atoms occurring in the intermediates or final compounds. Isotopes include those atoms having the same atomic number but different mass numbers. For example, isotopes of hydrogen include tritium and deuterium.

In some embodiments, the compounds of the disclosure, and salts thereof, are substantially isolated. By “substantially isolated” is meant that the compound is at least partially or substantially separated from the environment in which it was formed or detected. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or salt thereof, as would be understood by a person having ordinary skill in the art.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES. As used herein, with respect to measurements, “about” means+/−5%. As used herein, a recited ranges includes the end points, such that from 0.5 mole percent to 99.5 mole percent includes both 0.5 mole percent and 99.5 mole percent.

### EXAMPLES

The discussion of FIGS. 1-3B is supplemented with discussion of techniques and materials in reference to the following examples. It is understood that the examples described are intended to be illustrative rather than limiting, being provided to demonstrate the effectiveness and technical advantages and/or improvements of embodiments of the present disclosure over the current state of the art, instead of describing the scope of the present disclosure.

### Example 1: Photopatterned Biomolecule Immobilization to Guide 3D Cell Fate in Natural Protein-Based Hydrogels

In this Example, a generalizable strategy is presented for covalently decorating naturally derived biomaterials with bioactive proteins including growth factors so as to spatially control encapsulated cell fate. Taking advantage of primary amines 405 ubiquitously present on proteins (both at their N-terminus and on lysine side chains), natural gel precursors were minimally functionalized with a 2-(2-nitrophenyl) propoxycarbonyl (NPPOC)-photocaged alkoxyamine (H₂NO—) 410 through reaction with an activated N-Hydroxysuccinimide ester (—OSu), a chemistry chosen due to its comparatively long-term reagent stability, synthetic accessibility, and validated use for installing functional handles onto proteins. For an illustration of the functionalization reaction, refer to reaction scheme 400 of FIG. 4A.

The novel trifunctional NPPOC—HNO—OSu small molecule 415 (formula I) can be produced on gram scale through readily accessible chemistry, as described in more detail in reference to Example 2 and can be used to modify a wide variety of natural biomaterials. Upon exposure of the functionalized gel to cytocompatible near-UV radiation (λ=365 nm), the photocage (formula XVI) is cleaved in a dose-dependent manner, liberating the alkoxyamine (formula XXVIII) and permitting localized condensation with aldehyde-functionalized proteins, as illustrated in reactions schemes 430 and 450 of FIGS. 4B-4C. Through mask-based and laser-scanning lithographic activation of this photomediated oxime ligation, full 4D control over protein immobilization within three distinct natural biopolymer-based hydrogels was achieved. Highlighting the versatility of the approach, two responses are patterned that have not been achieved using purely synthetic materials, namely 2D primary rat hepatocyte proliferation on collagen gels using immobilized epidermal growth factor (EGF) and 3D U2OS Notch signaling activation within fibrin gels decorated with tethered Delta-1.

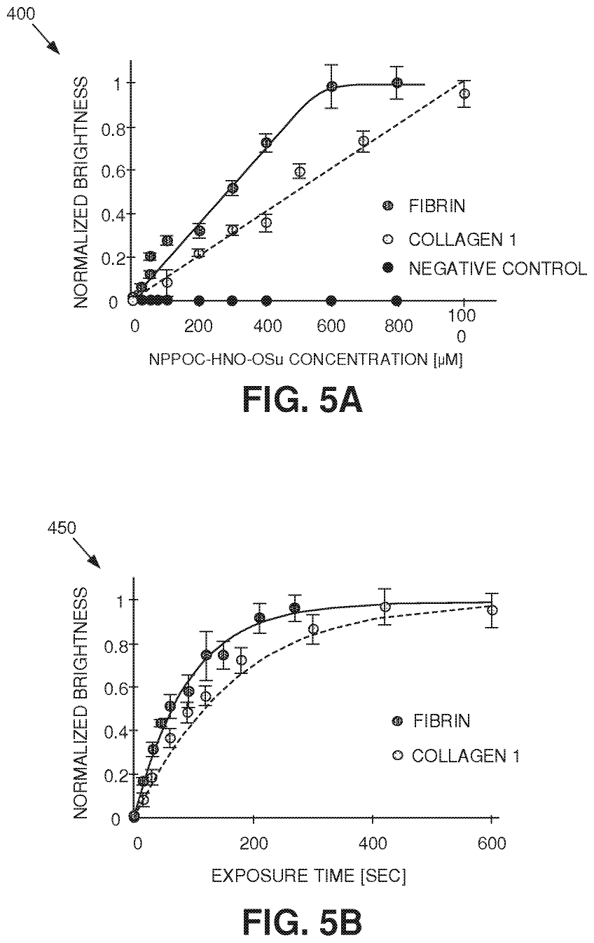

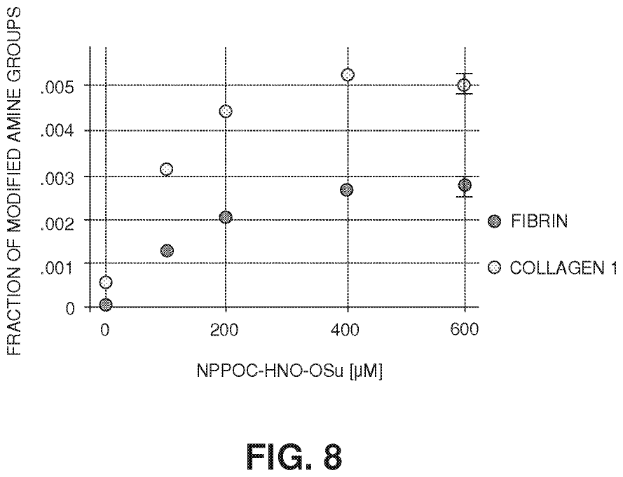

Workflow compatibility with different natural biomaterials was verified by biochemically decorated gels based on collagen I (4 mg mL⁻¹) or fibrin (10 mg mL⁻¹) with full-length proteins. Prior to gelation via conventional methodologies, collagen I and fibrin gel precursors were incubated with varying amounts of NPPOC—HNO—OSu 415 (0-1 mM) so as to create constructs bearing different concentrations of the photocaged alkoxyamine 410. Through theoretical calculations and experimental measurements, it was found that well under 1% of collagen and fibrin's primary amines were modified with the photocaged alkoxyamine following all treatment conditions; this small extent of labeling (<1 modification on average per protein) was anticipated to have minimal impact on ECM protein function, as described in more detail in reference to Examples 3-5 and illustrated in FIG. 8. For each protein-based gel system, transparent hydrogels were formed in cylindrical molds (diameter=3.2 mm, height=1 mm) and exposed to near-UV radiation (λ=365 nm, 10 mW cm⁻², 10 min). Based on NPPOC photo-uncaging kinetics, irradiation conditions were expected to yield complete alkoxyamine photo-liberation in a manner that is fully cytocompatible. After irradiation, gels were swollen in a solution containing an aldehyde-functionalized mCherry 465 (mCherry-CHO) (Sequence ID 2), a red-fluorescent protein site-specifically modified with the reactive carbonyl at its C-terminus through sortase-tag enhanced protein ligation (STEPL), as described in more detail in reference to FIG. 2 and Example 5. After gel-protein conjugation by oxime ligation, gels were washed to remove unbound protein prior to analysis. Taking advantage of the patterned protein's inherent fluorescence, the extent of mCherry 465 immobilization was quantified via fluorescent confocal microscopy (FIG. 5A). A linear correlation was observed between the concentration of NPPOC—HNO—OSu 415 and the total amount of protein phototethered within each gel, indicated by brightness in fluorescence microscopy. The variance in patterning slope between the collagen I and fibrin systems was attributed to differences in the total amine content of the gel precursors, resulting in a scaled functionalization for a given activated ester concentration. Control gels reacted with any amount of NPPOC—HNO—OSu 415 but never exposed to light yielded no mCherry 465 immobilization, indicating that the alkoxyamine remained fully caged throughout gel formation and successfully inhibited protein ligation, and that nonspecific protein fouling of the gel did not occur.

Following the demonstration that a synthetic workflow could be used to functionalize natural gels with full-length proteins, the effects of different irradiation conditions on gel patterning were evaluated. Hydrogels based on fibrin or collagen I were modified with NPPOC—HNO—OSu 415 (500 μM and 300 μM, respectively), cast as thin cylindrical constructs, and exposed to near-UV radiation (λ=365 nm, 10 mW cm⁻²) for varied amounts of time (0-10 min). Following incubation with mCherry-CHO 465 (0.5 mg mL⁻¹) and fluorescent quantification by confocal microscopy, an exponential dose-dependent response was observed as predicted by NPPOC photocleavage kinetics (FIG. 5B). Specifically, the NPPOC photodeprotection process was found to follow first-order reaction kinetics with decay constants of k=0.011±0.002 sec-1 in fibrin and 0.007±0.001 sec-1 in collagen I for λ=365 nm at 10 mW cm⁻². The minor variation in dose responsiveness between the two gel systems was attributed to subtle differences in the chemical microenvironment within each protein that yielded slightly varied photo-uncaging kinetics.

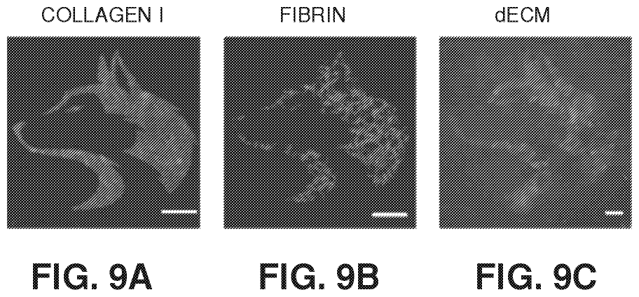

After demonstrating dose-dependent immobilization of full-length proteins in natural materials using flood illumination, mask-based lithographic techniques were employed to pattern NPPOC uncaging in arbitrary 2D geometries extending throughout the full thickness of each gel. Successful pattering was again observed in collagen I- and fibrin-based systems (FIGS. 9A-B), as indicated by the spatially controlled immobilization of mCherry-CHO 465 in the shape of a dog within gels, as illustrated in FIG. 3A. Similar results were observed for studies involving gels derived from decellularized cardiac extracellular matrix (ECM), as illustrated in FIG. 9C. Micron-scale patterning resolution was achieved in each material type, with patterning results in fibrin and dECM reflecting the fibrous structure characteristic to these natural materials.

While photomask-based lithography is readily employed to immobilize proteins within large gel volumes in a geometrically regulated and scalable manner, such methods are limited in that patterns can be specified in only two of the three spatial dimensions (i.e., x-y, but not in z). To that end, multiphoton laser-scanning lithographic patterning is an effective technique to locally modify user-defined hydrogel subvolumes with 4D control. Using these methodologies, photochemical reactions were confined to the focal point of a femtosecond-pulsed laser with high precision (sub-micron control over activation in x and y dimensions; 2-3 μm in z); raster scanning of the laser focal point within the sample results in localized material alteration in custom geometries, as described in more detail in reference to FIG. 2-3B. Such lithographic techniques were employed to immobilize mCherry-CHO 465 within NPPOC—HNO—OSu-functionalized collagen I- and fibrin-based gels in arbitrary shapes. Controlling the laser raster pattern in the x and y dimension, and stepping this activation through thin z-sections, 2D “slice” patterns were generated in shapes. Utilizing different laser scan 2D geometries at each z-location, fully 3D patterns were created in the shape of an anatomically correct human heart (FIG. 3B). Uniform 3D patterning with micron-scale patterning resolution was observed for both natural protein-based gel systems (collagen and fibrin).

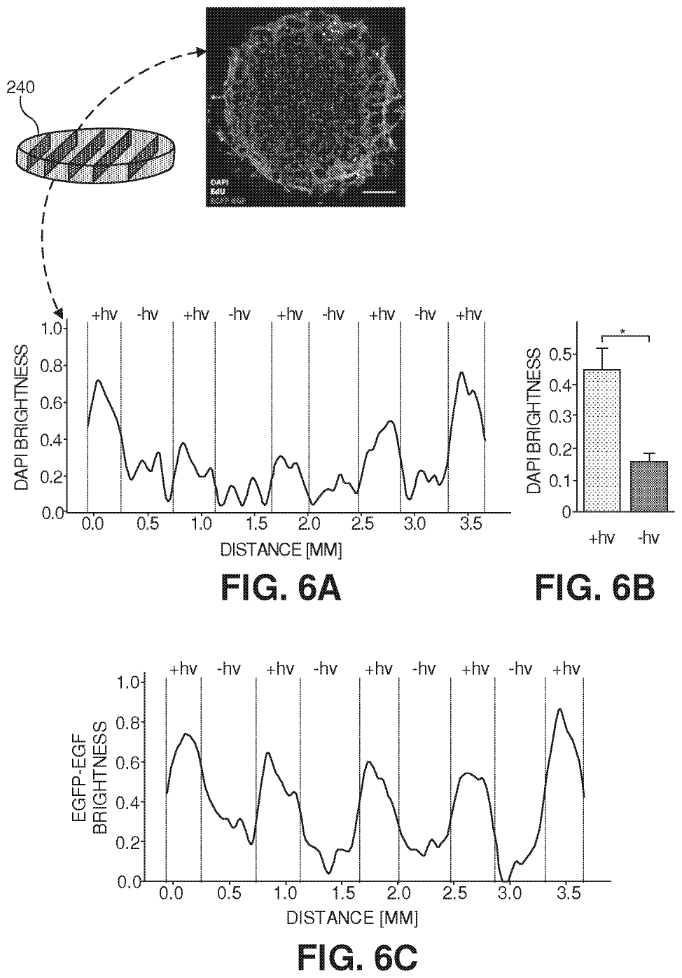

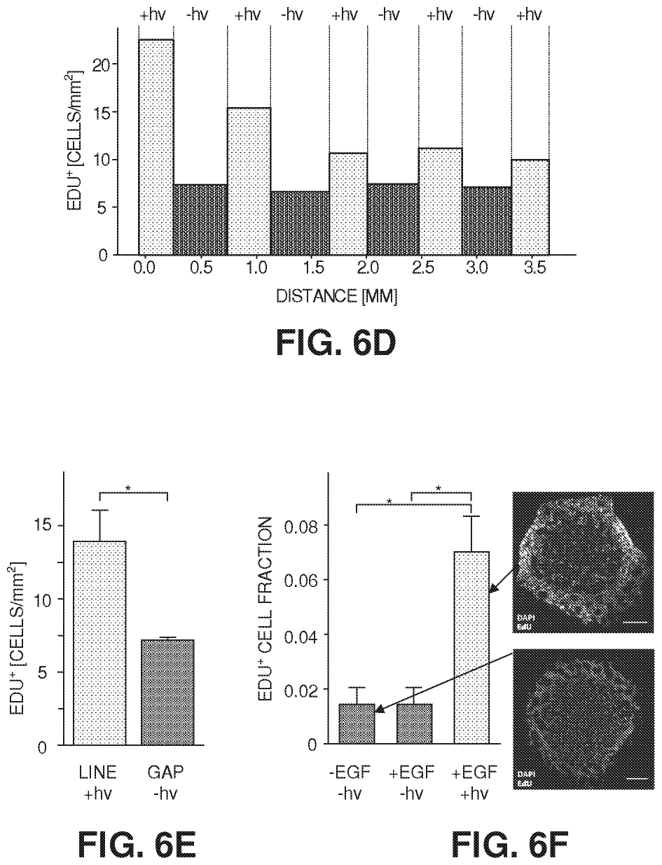

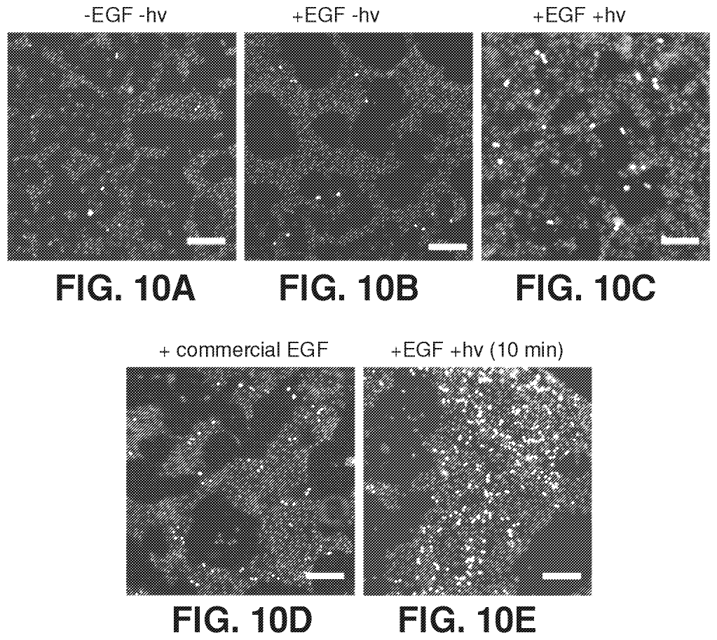

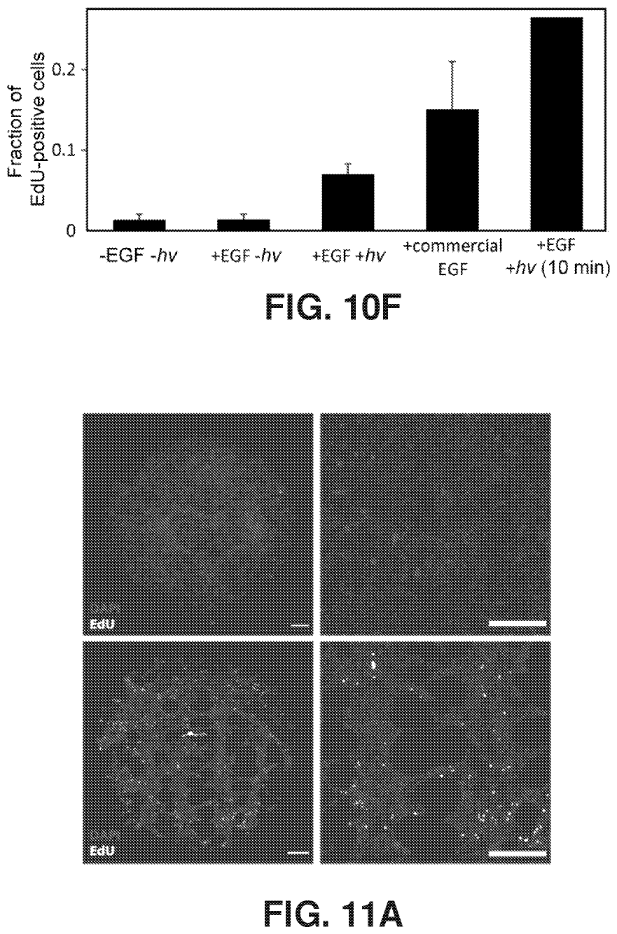

Following demonstration of site-specific immobilization of modified fluorescent proteins within natural hydrogel biomaterials, techniques described herein were employed to spatially direct complex biological fates using growth factors. Initially, epidermal growth factor (EGF) was targeted, which is a bioactive protein that stimulates proliferation of many cell types, including primary hepatocytes isolated from liver. Collagen I gels modified with NPPOC—HNO—OSu 415 (250 μM) were photochemically decorated with an aldehyde-functionalized EGF (EGF-CHO) that had been site-specifically tagged using STEPL, described in sequence listing (Sequence Listing ID 3). Freshly harvested primary rat hepatocytes seeded on EGF-modified gel surfaces exhibited a statistically significant increase in DNA synthesis (p<0.001, one-way ANOVA with Tukey's post-hoc) after 6 days in culture relative to control gels lacking EGF (FIG. 6F and FIGS. 10A-F), indicating that the tethered EGF remained bioactive.

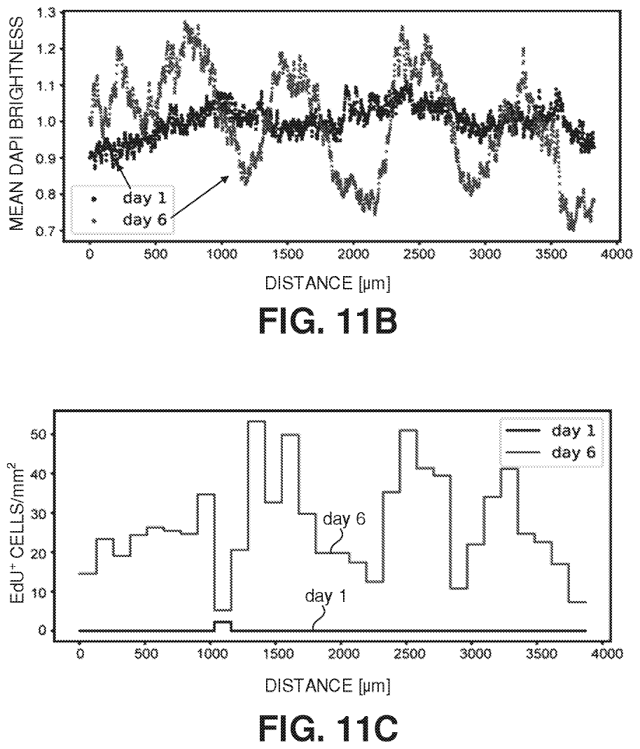

Mask-based lithography was used to photo-pattern collagen gels with lines (500 μm wide) of EGFP-EGF-CHO (Sequence Listing ID 4), a chimeric fusion of enhanced green fluorescent protein and the STEPL-tagged EGF-CHO that could be fluorescently visualized after immobilization. Samples were imaged after one and six days in culture on the protein-patterned surfaces, where non-uniform hepatocyte coverage was observed on the patterned surfaces only at the later timepoint; cell density and the number of cells synthesizing DNA indicated by EdU incorporation/staining were statistically higher on Day 6 (p=0.023 for DAPI, p=0.039 for EdU, t-test) but not day 1 in gel regions functionalized with EGFP-EGF-CHO compared with those without the immobilized growth factor (FIG. 6A-E and FIGS. 11A-C).

This study is significant for several reasons: (1) it is believed that this to be the first demonstration of patterned regulation of any primary cell type using a photoactivatable hydrogel, attributed to difficulty typically associated with culturing such cells on/in synthetic gels; (2) hepatocytes are notoriously non-proliferative cells, particularly in vitro, and these results appear to be a promising step towards promoting their expansion in culture; and (3) utilization of the photomediated oxime ligation in conjunction with the site-specifically modified EGF allows it to remain bioactive, despite its identity as a fragile growth factor.

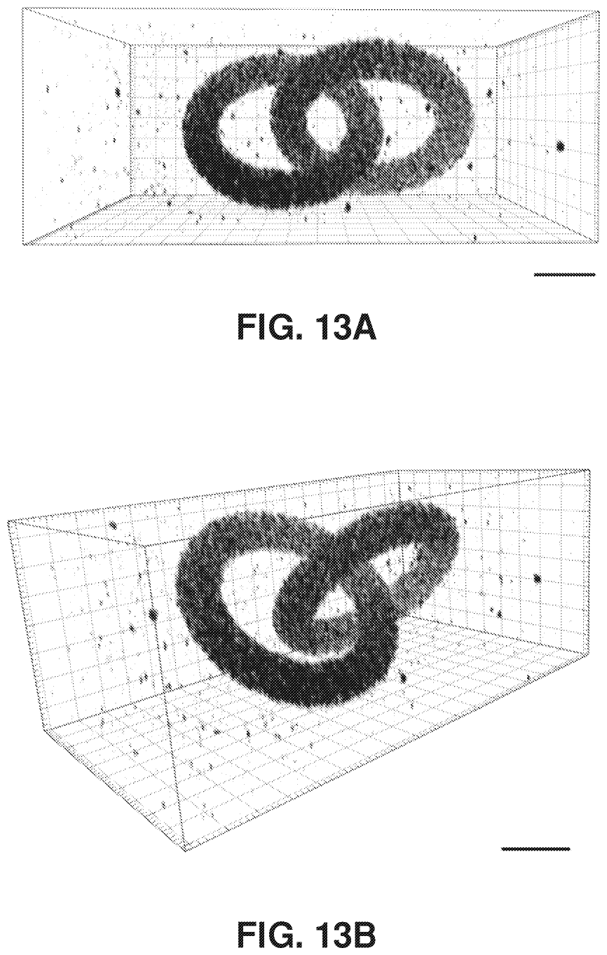

Having demonstrated the ability to direct cellular behavior in response to immobilized protein cues on gel surfaces, it was sought to exploit the inherent bioorthogonality of the photomediated oxime ligation to biochemically pattern natural hydrogels in the presence of encapsulated cells and to subsequently direct their fate in 3D. Specifically, Delta-1 protein 705 was patterned to activate the Notch 710 pathway (FIG. 7A), an evolutionary conserved signaling system required for normal embryonic development, regulation of tissue homeostasis, and maintenance of stem cells in adults that has proven unattainable in synthetic gels. Of the techniques typically employed to assess Notch 710 activation (e.g., RT-PCR, RNA microarray, Western blot analysis, with luciferase reporter cells), luciferase is the only method that can readily provide spatial readout, essential in evaluating gel pattering fidelity. Since luciferase activity cannot be easily resolved in three dimensions using conventional fluorescent imaging techniques (3D samples are imaged as 2D projections, as is done using IVIS imaging for in vivo analysis), 2D patterns of Delta-1 were prepared extending uniformly throughout the gel thickness (FIGS. 12A-12B). For these studies, recombinant Delta-1 705 was expressed in HEK293F mammalian cells, purified, and modified statistically with 2-5-dioxopyrrolidin-1-yl 4-formylbenzoate. This aldehyde-modified protein (Delta-1-CHO, sequence listing ID 5) was immobilized within cell-laden hydrogels in various geometries using mask-based photolithography. To quantify and visualize Notch 710 activation in response to patterned Delta-1 705, U2OS osteosarcoma reporter cells 715 expressing Renilla luciferase constitutively and firefly luciferase upon Notch 710 activation were encapsulated within fibrin gels. Whole-gel luminescent imaging on day 7 revealed a statistically significant (p<0.001, t-test) enhanced Notch 710 signaling in gel regions matching mask geometry (FIG. 7B-7F), as evidenced by higher firefly luciferase-catalyzed luminescence in the light-exposed biomaterial subvolumes. Luminescence originating from Renilla luciferase was constant for cells 715 throughout the entire gel (SI Appendix, Fig. S8), indicating that the protein patterning itself does not affect cell viability; these results demonstrate that the cellular proteome is unaltered in response to the near-UV treatments employed. Since full 3D control over Delta-1 705 immobilization within protein-based hydrogels 435 can be achieved using laser-scanning lithographic patterning (FIGS. 13A-13B), applications of the techniques described herein can include directing Notch and other complex signaling in 3D gels. In this way, the ability to spatially control complex cellular functions within the protein-based biomaterials most commonly exploited for 3D cell culture is a significant and powerful advance.

To that end, a synthetic workflow to immobilize bioactive proteins site-specifically and with spatiotemporal control within natural hydrogel biomaterials is provided. Employing a photomediated oxime ligation that is bioorthogonal and compatible with common lithographic patterning techniques, gel functionalization can be controlled with micron-scale resolution in a dose-dependent manner and in the presence of living cells. By infusing a new level of biochemical tunability into biomaterial platforms that have proven the modern workhorses of 3D cell and organoid culture, such approaches are expected to be applied in probing and directing biological functions and in engineering heterogenous functional tissues.

### Example 2: Synthesis of NPPOC—HNO—OSu

NPPOC—HNO—OSu was synthesized on gram scale via a two-step reaction from commercially available precursors with ˜80% overall yield. 2,5-dioxopyrrolidin-1-yl-2-((((2-(2-nitrophenyl)propoxy)carbonyl)amino)oxy) acetate (NPPOC—HNO—OSu) was synthesized following scale-up of the following synthetic route.

### Synthesis of 2-((((2-(2-nitrophenyl)propoxy)carbonyl)amino)oxy)acetic acid

Aminooxyacetic acid (4.23 g, 38.72 mmol) was dissolved in anhydrous dichloromethane (DCM, 100 mL) under argon. Triethylamine (12.64 μL, 96.8 nmol) was then added to the solution. 2,5-dioxopyrrolidin-1-yl (2-(2-nitrophenyl)propyl) carbonate[2] (6.24 g, 19.36 μmol) was dissolved in anhydrous DCM (50 mL), mixed with the aminooxyacetic acid solution, and stirred overnight protected from light under argon. The reaction product was concentrated under reduced pressure, re-dissolved in dH2O (200 mL), washed with ethyl acetate (EtOAc, 2×200 mL). The aqueous layer was acidified with HCl until pH reached 0. The product was extracted into EtOAc (3×200 mL), concentrated under reduced pressure, and purified via flash column chromatography (20:1 EtOAc:Acetic acid). The collected fractions were washed with water (3×200 mL), dried over MgSO4, filtered, and concentrated under reduced pressure to yield the pure product (4.52 g, 15.16 mmol, 78.3%). 1H NMR (500 MHz, CDCl3): 7.69-7.61 (m, 1H), 7.57-7.49 (m, 2H), 7.37-7.29 (m, 1H), 4.37-4.06 (m, 4H), 3.61-3.38 (m, 1H), 1.26 (d, J=7.05, 3H).

### Synthesis of 2,5-dioxopyrrolidin-1-yl-2-((((2-(2-nitrophenyl)propoxy)carbonyl)amino)oxy) acetate (NPPOC—HNO—OSu)

2-((((2-(2-nitrophenyl)propoxy)carbonyl)amino)oxy)acetic acid (0.95 g, 298.25 g/mol, 1×), N-hydroxysuccinimide (0.477 g, 115.09 g/mol, 1.3×), and EDC-HCl (0.794 g, 191.7 g/mol, 1.3×), were dissolved in anhydrous dimethylformamide (10 mL) in an N₂-purged flask and stirred overnight protected from light. The solvent was then removed under reduced pressure, and the product was redissolved in dichloromethane (100 mL). The organic layers were washed with water (3×100 mL), dried over MgSO4, filtered, and concentrated under reduced pressure to yield quantitatively the pure product (1.26 g, denoted NPPOC—HNO—OSu). 1H NMR (500 MHz, CDCl3): 7.98 (s, 1H), 7.76 (d. J=7.62, 1H), 7.59 (t, J=6.84, 1H), 7.47 (d, J=7.62, 1H), 7.39 (t, J=7.13, 1H), 4.70 (s, 1H), 4.39-4.24 (m, 4H), 3.82-3.64 (m, 1H), 2.99-2.68 (m, 4H), 1.37 (d, J=6.61, 3H).

### Example 3: Natural Protein-Based Hydrogel Functionalization with NPPOC—HNO—OSu

For collagen I functionalization: Collagen I (1 mL of rat tail collagen I at 4 mg mL⁻¹ in 0.02 N acetic acid, Corning) solution was brought to pH 7-7.5 on ice through the addition of phosphate buffered saline (PBS)-buffered NaOH, quickly mixed with NPPOC—HNO—OSu (25 mM in DMSO, 0-20 μL), and cast in molds. Formed gels were protected from light and washed in PBS overnight prior to patterning.

For fibrinogen functionalization: Stock fibrinogen solution (1 mL of 50 mg mL⁻¹ in Hank's balanced salt solution) was quickly mixed with NPPOC—HNO—OSu (25 mM in DMSO, 0-50 μL) and incubated (1 hour) at room temperature protected from light. To make gels, the functionalized fibrinogen solution was diluted to 10 mg mL⁻¹ through addition of PBS and thrombin (0.5-1.0 units mL⁻¹) prior to casting in molds. Formed gels were protected from light and washed in PBS overnight prior to patterning.

While illustrative embodiments have been illustrated and described, it will be appreciated that various changes can be made therein without departing from the spirit and scope of the disclosure.

### Example 4: Synthesis of Aldehyde-Modified Polyglycine Probe for STEPL

An aldehyde-modified polyglycine peptide was synthesized through Fmoc solid-phase methodologies involving a butyloxycarbonyl-protected N-terminal glycine and a 4-methyltrityl-protected C-terminal lysine reside. After selective deprotection of 4-methyltrityl [1% trifluoroacetic acid (TFA) in dichloromethane] on resin, aldehyde functionality was installed through condensation with 4-formylbenzoic and the ε-amino group of the C-terminal lysine. Resin was washed (dimethylformamide×3, and dichloromethane×3) prior to peptide cleavage/deprotection (95:5 TFA:H2O, 20 mL, 2 h) and precipitation (diethyl ether, 180 mL, 0° C., ×2). The crude peptide was purified via semi-preparative reversed-phase high-performance liquid chromatography using a 55-min gradient (5-100% of acetonitrile and 0.1% TFA in H2O) and lyophilized to give the final product [denoted H-GGGGDDK(CHO)—NH2]. Peptide purity was confirmed by matrix-assisted laser desorption/ionization time-of-flight mass spectrometry.

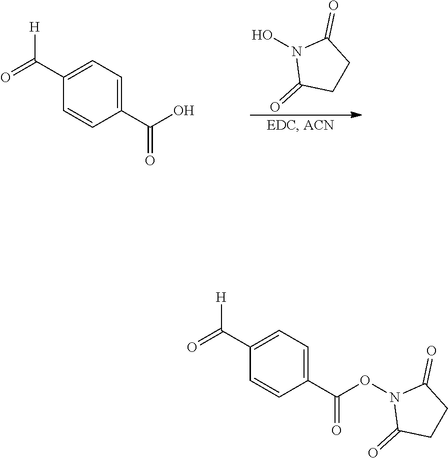 [SMILES: O=Cc1ccc(C(=O)O)cc1 ; O=C1CCC(=O)N1O ; O=Cc1ccc(C(=O)ON2C(=O)CCC2=O)cc1]

### Example 5: Aldehyde Functionalization of mCherry, EGF, and EGFP-EGF Proteins Via STEPL

STEPL plasmids for mCherry. EGF, and EGFP-EGF were constructed from pSTEPL37 using standard cloning techniques and transformed into BL21(DE3) Escherichia coli (Thermo Fisher). For protein expression, transformants were grown at 37° C. in a lysogeny broth that contained ampicillin (100 μg mL⁻¹) until an optical density of 0.6 (λ=600 nm). Expression was induced with isopropyl β-d-1-thiogalactopyranoside (0.5 mM) and then agitated overnight at 18° C. Cells were collected via centrifugation and lysed by sonication. The clarified lysate was loaded onto HisPur Ni-NTA resin (ThermoFisher), which was washed (20 mM Tris, 50 mM NaCl and 20 mM imidazole) to remove unbound proteins. After treatment of the resin with H-GGGGDDK(CHO)—NH2 (20×, 4 h, 37° C.) (Sequence ID 1), aldehyde-tagged proteins were eluted and purified by dialysis (molecular weight cut-off, ˜10 kDa). The protein identity and purity were confirmed by liquid chromatography-tandem mass spectrometry and sodium dodecyl sulfate polyacrylamide gel electrophoretic analysis. The protein concentrations were determined by UV absorption (λ=280 nm) prior to use. STEPL-modified proteins were denoted as mCherry-CHO, EGF-CHO, and EGFP-EGF-CHO.

### Example 6: Aldehyde Functionalization of Delta-1 Protein

Delta-1 protein was recombinantly expressed in HEK293F mammalian cells (ThermoFisher) and purified by immobilized metal affinity chromatography (SI Appendix, Method S4). 2-5-dioxopyrrolidin-1-yl 4-formylbenzoate (40 mM in DMSO, 5× molar excess) was added to Delta-1 (1 mg mL⁻¹ in PBS), incubated at room temperature (2 h), dialyzed in PBS at 4° C., and sterile filtered (0.2 μm syringe filter). This product (denoted Delta-1-CHO) was used without additional purification.

For Delta-1-IgG expression, Freestyle™ 293 Expression System (ThermoFisher) was used. For transient protein expression. HEK293F cells were transfected as per manufacturer's directions. Cells were cultured for 4 days, after which the medium was collected. To achieve stable transfection, cells at this stage were diluted to 2-5×10⁵ cells/mL and cultured in the presence of 150 μg mL⁻¹ G418 (geneticin, the concentration was chosen such that the non-transfected cells die within 2 weeks, while the transfected cells maintain positive growth rate) for 3 weeks with periodic passaging to maintain cell density below 10⁶ cells mL⁻¹. Subsequently, G418 concentration was reduced to 50 μg mL⁻¹ to increase cell proliferation rate. Cells were passaged in fresh medium supplemented with 50 μg mL⁻¹ G418 at 10⁶ cells mL⁻¹ and cultured in suspension for 4 days, after which the medium containing the expressed protein was collected.

Due to the natural affinity of Delta-1-IgG to nickel, we utilized immobilized metal affinity chromatography on Ni-NTA resin for its purification. 300 mL of medium was concentrated to <50 mL at 4° C. using a 50 mL Amicon® Stirred Cell with 10 kDa or 50 kDa molecular weight cutoff (MWCO) semi-permeable membrane. 10 mM imidazole and Ni-NTA resin was added to the medium and the solution was incubated under mild agitation for 2 hours at 4° C. The flow-through was discarded, and the resin was washed with wash buffer (20 mM Tris, 50 mM NaCl, 20 mM imidazole, 20 mL, 5×) at 4° C. The protein was eluted with ice-cold elution buffer (20 mM Tris, 50 mM NaCl, 500 mM imidazole, 1 mL, 10×) directly into 1 mL of 1M EDTA solution on ice. The protein was dialyzed in PBS and concentrated to >1 mg/mL by centrifugation at 4,000×g using a 10 kDa MWCO spin column at 4° C.

To introduce aldehyde functionality, 2-5-dioxopyrrolidin-1-yl 4-formylbenzoate (40 mM in DMSO, 5× molar excess) was added to the Delta-1-IgG solution, incubated for 2 hours at room temperature, dialyzed in PBS at 4° C., and sterile filtered (0.2 μm syringe filter). This product (denoted Delta-1-CHO) was used without additional purification. 2-5-dioxopyrrolidin-1-yl 4-formylbenzoate was synthesized via a known synthetic route. 4-formylbenzoic acid (2 g, 13.3 mmol, lx), N-Hydroxysuccinimide (1.69 g, 14.6 mmol, 1.1×), and N-(3-Dimethylaminopropyl)-N-ethylcarbodiimide hydrochloride (EDC HCl, 2.81 g, 14.6 mmol, 1.1×) were dissolved in anhydrous acetonitrile (50 mL) under argon. After stirring overnight at room temperature, the solvent was removed under reduced pressure, and the residue was resuspended in dichloromethane (100 mL). The organic layers were washed with water (3×), dried over MgSO₄, filtered, and concentrated to yield quantitatively the pure product (2,5-dioxopyrrolidin-1-yl 4-formylbenzoate, 3.29 g, 13.3 mmol) as a white solid. ¹H NMR (500 MHz, CDCl₃) δ 10.13 (s, 1H), 8.32-8.26 (m, 2H), 8.05-7.99 (m, 2H), 2.93 (s, 4H); ¹³C NMR (126 MHz, CDCl₃) δ 191.30, 169.08, 161.17, 140.45, 131.30, 130.08, 129.85, 25.80; HRMS (FAB+): calculated for C₁₂H₁₀NO₅+ [M+¹H]⁺, 248.0559; found 248.0569 (Δ=+4.1 ppm).

To fluorescently label Delta-1-IgG for visualization after photopatterning, Delta-1-IgG (1 mg mL⁻¹) was incubated (2 hr) with 5(6)-TAMRA-NHS Ester (10 molar excess, Click Chemistry Tools) at room temperature and dialyzed in PBS at 4° C. This product, denoted Delta-1-TAMRA-CHO, was used without additional purification.

### Example 7: Plasmid Construction for Protein Expression

Sources of DNA sequences: the pSTEPL plasmid containing EGFP and an ampicillin resistance gene was generously donated by the Tsourkas group (University of Pennsylvania). Plasmid containing gene sequence for mCherry was donated by the Baneyx group (University of Washington). EGF-bio-His was a gift from Gavin Wright (Addgene plasmid #53340). The Delta-1-IgG plasmid [pcDNA 3.1(+)] containing ampicillin and G418/kanamycin/neomycin resistance genes was generously donated by the Bernstein group (Fred Hutchinson Cancer Research Center).

STEPL plasmid construction: for each protein to be expressed in the STEPL system, polymerase chain reaction (PCR) was used to amplify gene sequences of interest and introduce relevant restriction sites (5′ NdeI and 3′ XhoI) for subsequent cloning. pSTEPL plasmid and PCR products were digested (4 hr, 37° C.) with NdeI and XhoI restriction enzymes (New England BioLabs) and purified by extraction following electrophoretic separation (0.8% agarose). The digested pSTEPL and protein insert were ligated (T4 DNA ligase, 16 hr, 16° C.), and transformed into chemically competent Top10 E. coli (Thermo Fisher) by heat shock, and plated onto agar plates (10 g Tryptone, 5 g Yeast Extract, 10 g NaCl, 15 g agar, 1 L dH₂O) containing ampicillin (100 μg mL⁻¹). Colonies were subsequently grown overnight in Miller's Lysogeny Broth (LB, 5 mL) containing ampicillin (100 μg mL⁻¹). Plasmids were purified using a QIAprep Spin Miniprep Kit (Qiagen), and sequenced using a SimpleSeq DNA Sequencing Kit (Thermo Fisher). Plasmids corresponding to the STEPL construct of interest were purified and subsequently transformed into chemically competent BL21(DE3) E. coli (Promega) for expression.

Delta-1-IgG plasmid expansion. For the Delta-1-IgG plasmid expansion to amounts used for mammalian cell transfection, it was transformed into chemically competent Top10 E. coli (Thermo Fisher) by heat shock, and plated onto agar plates (10 g Tryptone, 5 g Yeast Extract, 10 g NaCl, 15 g agar, 1 L dH2O) containing ampicillin (100 μg mL⁻¹). Colonies were subsequently grown overnight in Miller's Lysogeny Broth (LB, 50 mL) containing ampicillin (100 μg mL⁻¹). Plasmids were purified using a PureLink™ HiPure Plasmid Midiprep Kit (Thermo Fisher).

DNA open reading frame sequences: Sequence listing entries 2-5 list DNA open reading frame sequences (5′→3′) generated for and utilized in these studies. Nucleotides shown in black correspond to that for the specific protein of interest. Bases shown in orange correspond to STEPL portion common to all constructs, which features the C-terminal LPETG sortase recognition motif, a flexible (GGS)5 linker, SrtA, and a 6×His tag.

### Example 8: Hydrogel Patterning and Imaging

For photomask patterning, NPPOC—HNO—OSu-modified natural hydrogels were exposed to collimated UV radiation (λ=365 nm, 10 mW cm⁻², 0-10 min) through a patterned chrome photomask (Photo Sciences) using a Lumen Dynamics OmniCure S1500 Spot UV Curing system equipped with an internal 365 nm band-pass filter and a second in-line 360 nm cut-on long-pass filter. For the multiphoton laser-scanning lithography patterning experiments, an Olympus FV1000 MPE BX61 Multi-photon Microscope with a 20× objective was used. Gels were scanned in x-y scanned regions of interest (ROI) at different z positions throughout the gel thickness. Each 2D ROI was scanned 16-63 times with pulsed laser light (λ=740 nm, 0-100 laser power) with a 1.2 μm z interval to generate 3D patterns. After NPPOC cleavage, gels were incubated for ˜10 h with the aldehyde-tagged protein (0-100 μM in PBS) at 4° C. and protected from light. To wash away any unreacted protein, gels were gently agitated in PBS (16 h). Experiments involving patterned immobilization of fluorescent proteins were visualized by fluorescent, confocal, or multiphoton microscopy using standard imaging parameters.

### Example 9: Modulating Hepatocyte Survival and Proliferation Rate in Response to Patterned EGF

Collagen I gels (30 μL, 2 mg mL-1) were functionalized with NPPOC—HNO—OSu (250 μM) as previously described. Gels were subsequently exposed to UV radiation λ=365 nm, 10 mW cm⁻², 10 min), either directly or through a chrome mask patterned with 500 μm wide lines. Gels were then incubated with EGF-CHO or EGFP-EGF-CHO (0.1 mg mL⁻¹ in Tris buffer, 10 h) prior to extensive washing in PBS (3×, 8 h). Primary hepatocytes were isolated from female Lewis/SsNHsd rats (Envigo), then seeded (80,000 cells cm-2) on gels in Dulbecco's Modified Eagle Medium (supplemented with 10% Fetal Bovine Serum, 1% Penicillin-Streptomycin, 15 mM HEPES, 0.04 μg mL⁻¹ dexamethazone, 70 μg mL⁻¹ glucagon, and 1% insulin-transferrin-sodium selenite supplement, Sigma-Aldrich). Cells were cultured for 6 days with media changes on days 1, 3, 4, and 5. On days 4 and 5 media was supplemented with 5-ethynyl-2′-deoxyuridine (EdU, 1:1000, Invitrogen). After 1 and 6 days of culture, samples were fixed (4% paraformaldehyde) prior to staining for nuclei (Hoechst 33342, 1:2000) and EdU (Click-iT EdU Imaging Kit, Invitrogen C10340). Whole samples were fluorescently imaged (Nikon Eclipse Ti high-resolution fluorescent widefield imaging system). The fraction of EdU-positive nuclei was determined using a custom MATLABX script measuring the ratio of EdU-positive to Hoechst-positive pixels for each image. Line/gap edges was ascribed to low-high intensity transitions in the EGFP-EGF-CHO fluorescence. The line/gap cell density was assessed for patterned gels by measuring the mean nuclei fluorescence along the direction of the line. EdU+ cell analysis was performed with the aid of a custom MATLAB segmentation algorithm which detected and determined locations of EdU+ nuclei.

### Example 10: Patterned Notch Activation of U2OS Cells in Response to Immobilized Delta-1

U2OS osteosarcoma CSL/luciferase Notch reporter cells (10 cell mL⁻¹) were encapsulated in collagen I gels (30 μL, 10 mg mL-1) functionalized with NPPOC—HNO—OSu (250 μM). Cell-laden gels were cast in the presence of Delta-1-CHO (0.05 mg mL⁻¹) and irradiated with light (λ=365 nm, 10 mW cm⁻², 10 min) through various photomasks (half on; array of W's; 500 μm wide lines) to pattern aminooxy uncaging. 2 hours after light exposure, gels were placed in and washed with medium (Dulbecco's Modified Eagle Medium supplemented with 10% Fetal Bovine Serum, 1% Penicillin-Streptomycin, 2 μg mL⁻¹ aprotinin; changed daily for 6 days). Luciferase expression was determined on day 7 using a Promega Dual-Glo® Luciferase Assay per manufacturer instruction, with D-luciferin (150 μg mL⁻¹) added to the medium prior to analysis. Gels were imaged using a ChemiDoc XRS+ (BioRad) and In Vivo Imaging System Spectrum (PerkinElmer IVIS®). Overlaying the aligned photomask image file onto each of the patterned gels, Notch activation was quantified for cells within the Delta-modified gel subvolumes and compared with those in the unfunctionalized regions. Statistical analyses were performed using a standard t-test.

1. A heterobifunctional linker, comprising:
a peptide-reactive activated functional group on the heterobifunctional linker, comprising an activated amine-reactive functional group, an activated thiol-reactive functional group, or any combination thereof, wherein the peptide-reactive activated functional group is reactive with a hydrogel comprising a crosslinked natural polymer, and
a photocaged reactive group on the heterobifunctional linker, comprising a photocaged hydroxylamine, a photocaged alkoxyamine, a photocaged hydrazide, a photocaged amine, a photocaged tetrazine, or a photocaged alkyne-containing moiety,
wherein the peptide-reactive activated functional group does not comprise an azide.

2. The heterobifunctional linker of claim 1, wherein the peptide-reactive activated functional group comprises a N-hydroxysuccinimidide (NHS) activated amine-reactive functional group (e.g., an NHS ester), a N-hydroxysulfosuccinimido (NHSS) activated amine-reactive functional group (e.g., a NHSS ester), an isocyanate, an isothiocyanate, or a maleimide-activated thiol-reactive functional group.

3. The heterobifunctional linker of claim 1, wherein the photocaged reactive group is adapted to be uncaged to provide the reactive group, following exposure to electromagnetic radiation having a predetermined wavelength to provide a reactive hydroxylamine, an alkoxyamine, a reactive hydrazide, a reactive amine, a reactive tetrazine, or a reactive alkyne.

4. The heterobifunctional linker of claim 1, wherein the photocaged reactive group comprises a reactive group caged with an optionally substituted 2-(2-nitrophenyl) propoxycarbonyl moiety, an optionally substituted nitrobenzyl moiety, an optionally substituted coumarin moiety, an optionally substituted boron-dipyrromethene (BODIPY) moiety, or an optionally substituted ruthenium complex.

5. A heterobifunctional linker, having a structure: 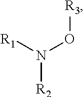
wherein R₁ is selected from a group consisting of an optionally substituted 2-(2-nitrophenyl) propoxycarbonyl moiety, an optionally substituted nitrobenzyl moiety, an optionally substituted coumarin moiety, an optionally substituted boron-dipyrromethene (BODIPY) moiety, and an optionally substituted ruthenium complex;
wherein R₂ is selected from a group consisting of hydrogen, an alkane, an alkyl, and an alcohol; and
wherein R₃ is selected from a group consisting of a N-hydroxysuccinimidide (NHS) activated amine-reactive functional group (e.g., an NHS ester), a N-hydroxysulfosuccinimido (NHSS) activated amine-reactive functional group (e.g., a NHSS ester), an isocyanate, an isothiocyanate, or a maleimide-activated thiol-reactive functional group.

6. The heterobifunctional linker of claim 5, wherein R₁ has a structure: 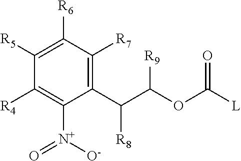
wherein “L” represents the binding position for the structure of claim 5;
wherein R₄-R₉ are independently selected from a group consisting of methyl, methoxy, carboxylic acid, ethyl, ethoxy, alkyl, aryl, —OH, —OR, —OC₆H₅, —NH₂, —NR₂, —NHCOCH₃, —CH₂Cl, —F, —Cl, —Br, —I, —CH═CHNO₂, —NO₃, —NH₃+, —PR₃+, —SR₂, —IC6H₅, —CF₃, —CCl₃, —SO₃H, —SO₂R, —CO₂H, —CO₂R, —CONH₂, —CHO, —COR, and —CN; and
wherein the combination of substitutions at R₄-R₉ determines a wavelength of electromagnetic radiation at which R₁ is removed from the structure of claim 5.

7. The heterobifunctional linker of claim 5, wherein R₃ has a structure: 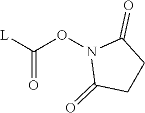
wherein “L” represents the binding position for the structure of claim 5.

8. The heterobifunctional linker of claim 5, having the structure:

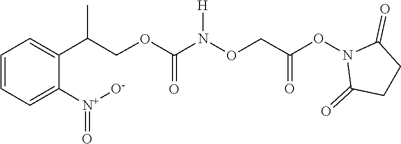 [SMILES: CC(COC(=O)NOCC(=O)ON1C(=O)CCC1=O)c1ccccc1[N+](=O)[O-]]

9. The heterobifunctional linker of claim 5, having the structure:

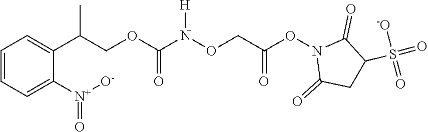

10. The heterobifunctional linker of claim 5, having the structure:

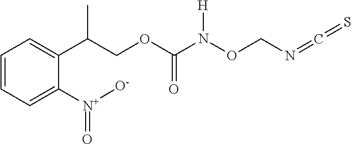 [SMILES: CC(COC(=O)NOCN=C=S)c1ccccc1[N+](=O)[O-]]

11. The heterobifunctional linker of claim 5, having the structure:

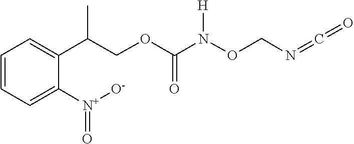 [SMILES: CC(COC(=O)NOCN=C=O)c1ccccc1[N+](=O)[O-]]

12. The heterobifunctional linker of claim 5, having the structure:

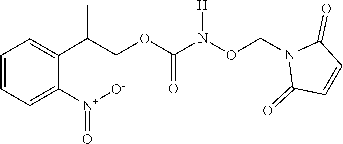 [SMILES: CC(COC(=O)NOCN1C(=O)C=CC1=O)c1ccccc1[N+](=O)[O-]]

13. The heterobifunctional linker of claim 5, having the structure:

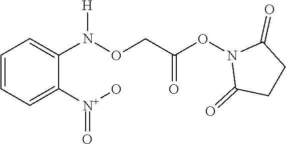

14. The heterobifunctional linker of claim 5, having the structure:

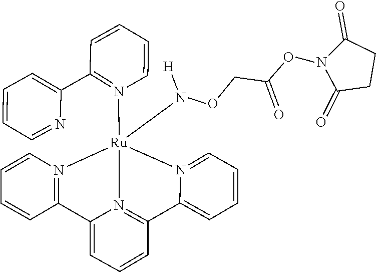

15. The heterobifunctional linker of claim 5, having the structure: 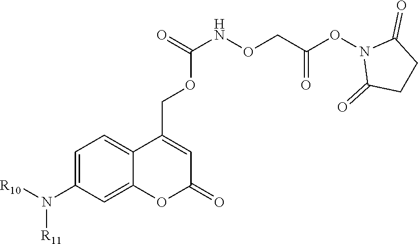
wherein R10 and R11 are independently selected from a group consisting of hydrogen, alkane, alkyl, carboxyl, or the like.

16. The heterobifunctional linker of claim 5, having the structure: 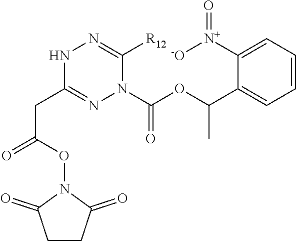
Wherein R₁₂ is selected from a group consisting of Hydrogen, an alkane, an alkyl, an alcohol, an alkyne, and a methyl.

17. The heterobifunctional linker of claim 16, having the structure:

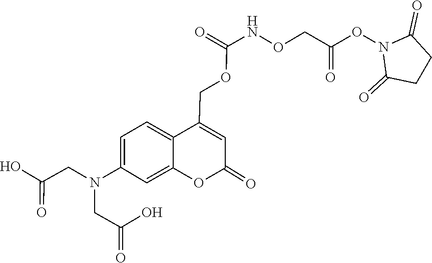 [SMILES: C=C/C=C(\C=C1/CC(COC(=O)NOCC(=O)ON2C(=O)CCC2=O)=CC(=O)O1)N(CC(=O)O)CC(=O)O]

18. The heterobifunctional linker of claim 16, having the structure:

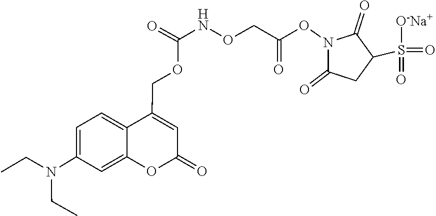

19. A hydrogel, comprising:
a hydrogel matrix comprising a crosslinked natural polymer; and
a covalently linked hydrogel label at predetermined locations in the hydrogel matrix, wherein the hydrogel label is covalently linked to the hydrogel via a linker.

20-38. (canceled)
